(19) United States
(12) Patent Application Publication
(10) Pub. No.: US 2009/0039765 A1
(43) Pub. Date: Feb. 12, 2009

(54) LIGHT EMITTING POLYMER COMPOSITION AND POLYMER LIGHT EMITTING DEVICE

(76) Inventors: Yasunori Uetani, Ibaraki (JP); Nobuhiko Shirasawa, Kanagawa (JP); Hirotoshi Nakanishi, Osaka (JP)

Correspondence Address: SUGHRUE MION, PLLC, 2100 PENNSYLVANIA AVENUE, N.W., SUITE 800, WASHINGTON, DC, 20037, US

(21) Appl. No.: 11/574,029

(22) PCT Filed: Aug. 23, 2005
(86) PCT No.: PCT/JP05/15606

§ 371 Date: Aug. 21, 2007

(30) Foreign Application Priority Data
Aug. 31, 2004 (JP) 2004-251725
Nov. 19, 2004 (JP) 2004-335575

(51) Int. Cl. H01J 1/62; C09K 11/06; C07D 209/82

(52) U.S. Cl. 313/504; 252/301.35; 548/444

(57) ABSTRACT

A light emitting polymer composition comprising a light emitting polymer and a compound selected from the following formulae (1a) to (1d):

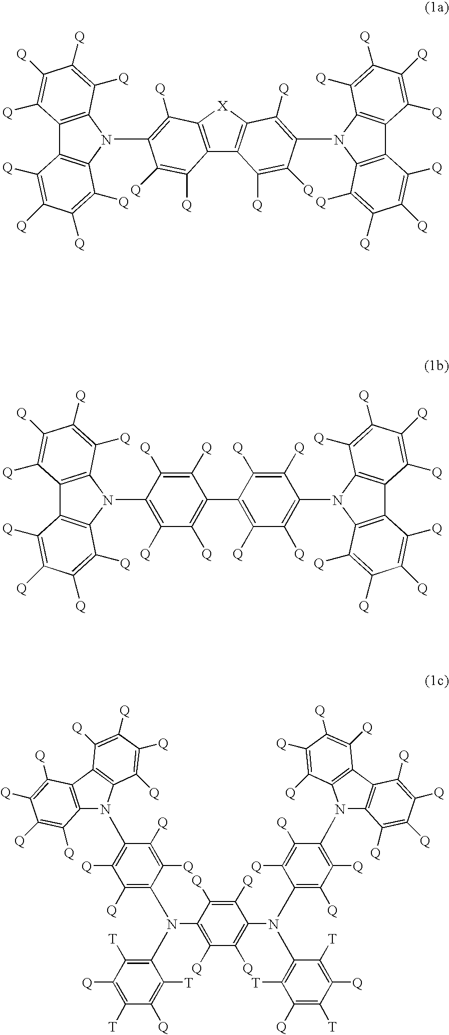

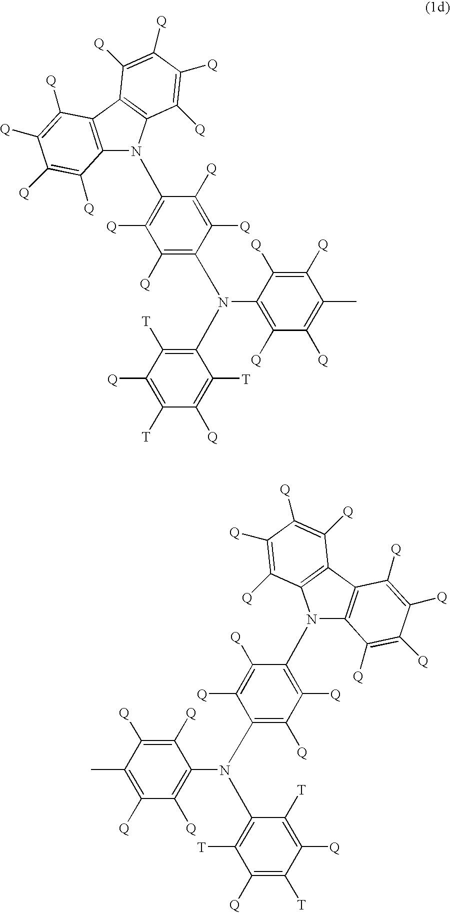

(wherein, X represents an atom or atomic group forming a 5-membered or 6-membered ring together with four carbon atoms on two benzene rings in the formula, Q and T represent each independently a hydrogen atom, halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, substituted silyloxy group, substituted silylthio group, substituted silylamino group, substituted amino group, amide group, acid imide group, acyloxy group, mono-valent heterocyclic group, heteroaryloxy group, heteroarylthio group, cyano group or nitro group).

### TECHNOLOGICAL FIELD

The present invention relates to a light emitting polymer composition, a light emitting polymer solution composition, and a polymer light emitting device (polymer LED) using the same.

### BACKGROUND ART

Light emitting polymers (light emitting materials of high molecular weight) are soluble in a solvent, differing from those of low molecular weight, thus, capable of forming a light emitting layer in a light emitting device by an application method, responding to a requirement of larger area of the device. Therefore, there are recently suggested various polymer light emitting materials (for example, Advanced Materials Vol. 12, 1737-1750 (2000)).

Here, light emitting devices are desired to show high light emitting efficiency, thus, high light emitting brilliance per electric current. However, when light emitting polymers are used, the device efficiency thereof is not satisfactory yet.

### DISCLOSURE OF THE INVENTION

The present invention has an object of providing a light emitting polymer composition which can impart a light emitting device of high efficiency when used in a light emitting layer of the light emitting device.

The present inventors have investigated to solve the above-described problem and resultantly found that when a composition having a compound of a specific structure contained in a light emitting polymer is used as a material of a light emitting layer of a light emitting device, a light emitting device of remarkably improved efficiency is obtained, leading to the present invention.

That is, the present invention provides a light emitting polymer composition containing a light emitting polymer and a compound selected from the following formulae (1a) to (1d):

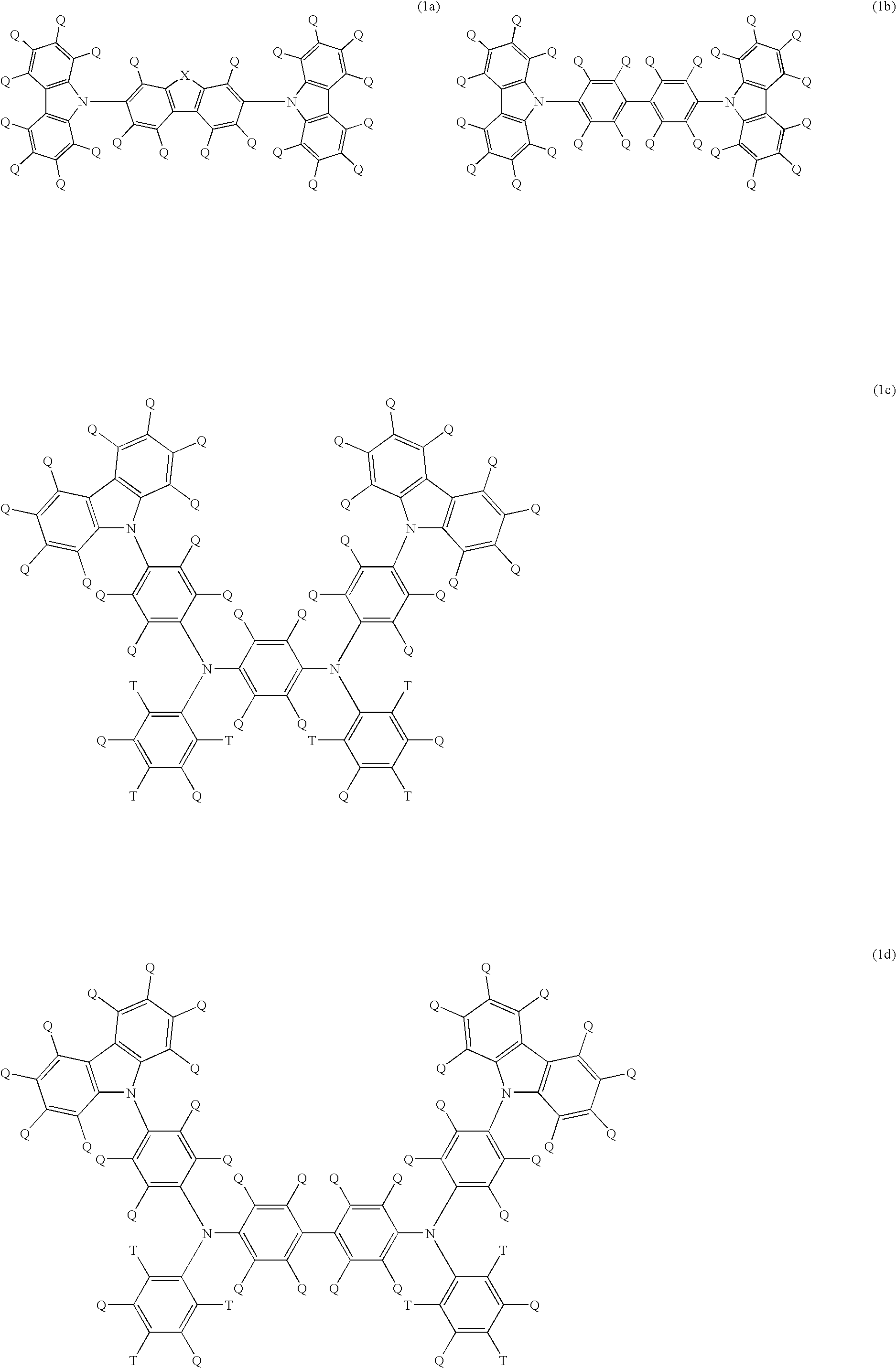

(wherein, X represents an atom or atomic group forming a 5-membered or 6-membered ring together with four carbon atoms on two benzene rings in the formula, Q and T represent each independently a hydrogen atom, halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, substituted silyloxy group, substituted silylthio group, substituted silylamino group, substituted amino group, amide group, acid imide group, acyloxy group, mono-valent heterocyclic group, heteroaryloxy group, heteroarylthio group, cyano group or nitro group, and of them, any two moieties bonding to adjacent carbon atoms may together form a ring).

Further, the present invention relates to a light emitting polymer solution composition containing the above-described light emitting polymer and a compound selected from the above-described formulae (1a) to (1d), and additionally, a solvent. Furthermore, the present invention relates to a compound of the above-described formula (1c).

### BEST MODES FOR CARRYING OUT THE INVENTION

The compound to be used in the composition of the present invention is represented by the above-described formulae (1a) to (1d).

As the halogen atom represented by Q and T in the formulae (1a) to (1d), exemplified are fluorine, chlorine, bromine and iodine.

The alkyl group may be any of linear, branched or cyclic, may have a substituent, and the total carbon number is usually 1 to about 20, and specific examples thereof include a methyl group, ethyl group, propyl group, i-propyl group, butyl group, i-butyl group, t-butyl group, pentyl group, hexyl group, cyclohexyl group, heptyl group, octyl group, 2-ethylhexyl group, nonyl group, decyl group, 3,7-dimethyloctyl group, lauryl group, trifluoromethyl group, pentafluoroethyl group, perfluorobutyl group, perfluorohexyl group, perfluorooctyl group and the like. The substituent includes halogens, oxetane group, epoxy group, oxetidinyl group, oxolidinyl group, oxolanyl group, oxanyl group, oxonanyl group, oxathioranyl group, piperidyl group and the like.

The alkyloxy group may be any of linear, branched or cyclic, may have a substituent, and the total carbon number is usually 1 to about 20, and specific examples thereof include a methoxy group, ethoxy group, propyloxy group, i-propyloxy group, butoxy group, i-butoxy group, t-butoxy group, pentyloxy group, hexyloxy group, cyclohexyloxy group, heptyloxy group, octyloxy group, 2-ethylhexyloxy group, nonyloxy group, decyloxy group, 3,7-dimethyloctyloxy group, lauryloxy group, trifluoromethoxy group, pentafluoroethoxy group, perfluorobutoxy group, perfluorohexyl group, perfluorooctyl group, methoxymethyloxy group, 2-methoxyethyloxy group and the like. The substituent includes halogens, oxetane group, epoxy group, oxetidinyl group, oxolidinyl group, oxolanyl group, oxanyl group, oxonanyl group, oxathioranyl group, piperidyl group and the like.

The alkylthio group may be any of linear, branched or cyclic, may have a substituent, and the total carbon number is usually 1 to about 20, and specific examples thereof include a methylthio group, ethylthio group, propylthio group, i-propylthio group, butylthio group, i-butylthio group, t-butylthio group, pentylthio group, hexylthio group, cyclohexylthio group, heptylthio group, octylthio group, 2-ethylhexylthio group, nonylthio group, decylthio group, 3,7-dimethyloctylthio group, laurylthio group, trifluoromethylthio group and the like. The substituent includes halogens, oxetane group, epoxy group, oxetidinyl group, oxolidinyl group, oxolanyl group, oxanyl group, oxonanyl group, oxathioranyl group, piperidyl group and the like.

The aryl group may have a substituent, and the total carbon number is usually about 3 to 60, and specific examples thereof include a phenyl group, C₁-C₁₂ alkoxyphenyl groups (C₁-C₁₂ means a carbon number of 1 to 12, applicable also in the followings), C₁-C₁₂ alkylphenyl groups, 1-naphthyl group, 2-naphthyl group, pentafluorophenyl group and the like. The substituent includes halogens, oxetane group, epoxy group, oxetidinyl group, oxolidinyl group, oxolanyl group, oxanyl group, oxonanyl group, oxathioranyl group, piperidyl group and the like.

The aryloxy group may have a substituent on an aromatic ring, and the total carbon number is usually about 3 to 60, and specific examples thereof include a phenoxy group, C₁-C₁₂ alkoxyphenoxy groups, C₁-C₁₂ alkylphenoxy groups, 1-naphthyloxy group, 2-naphthyloxy group, pentafluorophenyloxy group and the like. The substituent includes alkoxy groups, alkyl groups, halogens, oxetane group, epoxy group, oxetidinyl group, oxolidinyl group, oxolanyl group, oxanyl group, oxonanyl group, oxathioranyl group, piperidyl group and the like.

The arylthio group may have a substituent on an aromatic ring, and the total carbon number is usually about 3 to 60, and specific examples thereof include a phenylthio group, C₁-C₁₂ alkoxyphenylthio groups, C₁-C₁₂ alkylphenylthio groups, 1-naphthylthio group, 2-naphthylthio group, pentafluorophenylthio group and the like. The substituent includes alkoxy groups, alkyl groups, halogens, oxetane group, epoxy group, oxetidinyl group, oxolidinyl group, oxolanyl group, oxanyl group, oxonanyl group, oxathioranyl group, piperidyl group and the like.

The arylalkyl group may have a substituent, and the total carbon number is usually about 7 to 60, and specific examples thereof include phenyl-C₁-C₁₂ alkyl groups, C₁-C₁₂ alkoxyphenyl-C₁-C₁₂ alkyl groups, C₁-C₁₂ alkylphenyl-C₁-C₁₂ alkyl groups, 1-naphthyl-C₁-C₁₂ alkyl groups, 2-naphthyl-C₁-C₁₂ alkyl groups and the like.

The substituent includes alkoxy groups, alkyl groups, halogens, oxetane group, epoxy group, oxetidinyl group, oxolidinyl group, oxolanyl group, oxanyl group, oxonanyl group, oxathioranyl group, piperidyl group and the like.

The arylalkyloxy group may have a substituent, and the total carbon number is usually about 7 to 60, and specific examples thereof include phenyl-C₁-C₁₂ alkoxy groups, C₁-C₁₂ alkoxyphenyl-C₁-C₁₂ alkoxy groups, C₁-C₁₂ alkylphenyl-C₁-C₁₂ alkoxy groups, 1-naphthyl-C₁-C₁₂ alkoxy groups, 2-naphthyl-C₁-C₁₂ alkoxy groups and the like. The substituent includes alkoxy groups, alkyl groups, halogens, oxetane group, epoxy group, oxetidinyl group, oxolidinyl group, oxolanyl group, oxanyl group, oxonanyl group, oxathioranyl group, piperidyl group and the like.

The arylalkylthio group may have a substituent, and the total carbon number is usually about 7 to 60, and specific examples thereof include phenyl-C₁-C₁₂ alkylthio groups, C₁-C₁₂ alkoxyphenyl-C₁-C₁₂ alkylthio groups, C₁-C₁₂ alkylphenyl-C₁-C₁₂ alkylthio groups, 1-naphthyl-C₁-C₁₂ alkylthio groups, 2-naphthyl-C₁-C₁₂ alkylthio groups and the like. The substituent includes alkoxy groups, alkyl groups, halogens, oxetane group, epoxy group, oxetidinyl group, oxolidinyl group, oxolanyl group, oxanyl group, oxonanyl group, oxathioranyl group, piperidyl group and the like.

The alkenyl group has a carbon number of usually about 2 to 20, and specific examples thereof include a 1-propylenyl group, 2-propylenyl group, 3-propylenyl group, butenyl group, pentenyl group, hexenyl group, heptenyl group, octenyl group and cyclohexenyl group.

The alkenyl group includes also alkadienyl groups such as a 1,3-butadienyl group and the like.

The alkynyl group has a carbon number of usually about 2 to 20, and specific examples thereof include an ethynyl group, 1-propynyl group, 2-propynyl group, butynyl group, pentynyl group, hexynyl group, heptenyl group, octynyl group and cyclohexylethynyl group. The alkynyl group includes also alkydienyl groups such as a 1,3-butadiynyl group and the like.

The arylalkenyl group has a carbon number of usually about 8 to 50, and the aryl group and the alkenyl group in the arylalkenyl group are the same as the aryl group and alkenyl group described above, respectively. Specific examples thereof include a 1-arylvinyl group, 2-arylvinyl group, 1-aryl-1-propylenyl group, 2-aryl-1-propylenyl group, 2-aryl-2-propylenyl group, 3-aryl-2-propylenyl group and the like. Arylalkadienyl groups such as a 4-aryl-1,3-butadienyl group and the like are also included.

The arylalkynyl group has a carbon number of usually about 8 to 50, and the aryl group and the alkynyl group in the arylalkynyl group are the same as the aryl group and alkynyl group described above, respectively. Specific examples thereof include an arylethynyl group, 3-aryl-1-propionyl group, 3-aryl-2-propionyl group and the like. Arylalkadiynyl groups such as a 4-aryl-1,3-butadiynyl group and the like are also included.

The substituted silyl group in the substituted silyloxy group includes silyl groups substituted with 1, 2 or 3 groups selected from alkyl groups, aryl groups, arylalkyl groups and mono-valent heterocyclic groups, and the carbon number is usually 1 to about 60, preferably 3 to 30. The alkyl group, aryl group, arylalkyl group or mono-valent heterocyclic group may have a substituent. Specific examples thereof include a trimethylsilyloxy group, triethylsilyloxy group, tri-n-propylsilyloxy group, tri-i-propylsilyloxy group, t-butylsilyldimethylsilyloxy group, triphenylsilyloxy group, tri-p-xylylsilyloxy group, tribenzylsilyloxy group, diphenylmethylsilyloxy group, t-butyldiphenylsilyloxy group, dimethylphenylsilyloxy group and the like.

The substituted silyl group in the substituted silylthio group includes silyl groups substituted with 1, 2 or 3 groups selected from alkyl groups, aryl groups, arylalkyl groups and mono-valent heterocyclic groups, and the carbon number is usually 1 to about 60, preferably 3 to 30. The alkyl group, aryl group, arylalkyl group or mono-valent heterocyclic group may have a substituent. Specific examples thereof include a trimethylsilylthio group, triethylsilylthio group, tri-n-propylsilylthio group, tri-i-propylsilylthio group, t-butylsilyldimethylsilylthio group, triphenylsilylthio group, tri-p-xylylsilylthio group, tribenzylsilylthio group, diphenylmethylsilylthio group, t-butyldiphenylsilylthio group, dimethylphenylsilylthio group and the like.

The substituted silylamino group includes silylamino groups (H₃SiNH— or (H₃Si)₂N—) substituted with 1 to 6 groups selected from alkyl groups, aryl groups, arylalkyl groups and mono-valent heterocyclic groups, and has a carbon number of usually 1 to 120, preferably 3 to 60. The alkyl group, aryl group, arylalkyl group or mono-valent heterocyclic group may have a substituent. Specific examples thereof include a trimethylsilylamino group, triethylsilylamino group, tri-n-propylsilylamino group, tri-i-propylsilylamino group, t-butylsilyldimethylsilylamino group, triphenylsilylamino group, tri-p-xylylsilylamino group, tribenzylsilylamino group, diphenylmethylsilylamino group, t-butyldiphenylsilylamino group, dimethylphenylsilylamino group, di(trimethylsilyl)amino group, di(triethylsilyl)amino group, di(tri-n-propylsilyl)amino group, di(tri-i-propylsilyl)amino group, di(t-butylsilyldimethylsilyl)amino group, di(triphenylsilyl)amino group, di(tri-p-xylylsilyl)amino group, di(tribenzylsilyl)amino group, di(diphenylmethylsilyl)amino group, di(t-butyldiphenylsilyl)amino group, di(dimethylphenylsilyl)amino group and the like.

The substituted amino group includes amino groups substituted with one or two groups selected from alkyl groups, aryl groups, arylalkyl groups and mono-valent heterocyclic groups, and the alkyl group, aryl group, arylalkyl group or mono-valent heterocyclic group may have a substituent. The substituted amino group has a carbon number of usually 1 to about 40, and specific examples thereof include a methylamino group, dimethylamino group, ethylamino group, diethylamino group, propylamino group, dipropylamino group, isopropylamino group, diisopropylamino group, butylamino group, isobutylamino group, t-butylamino group, pentylamino group, hexylamino group, cyclohexylamino group, heptylamino group, octylamino group, 2-ethylhexylamino group, nonylamino group, decylamino group, 3,7-dimethyloctylamino group, laurylamino group, cyclopentylamino group, dicyclopentylamino group, cyclohexylamino group, dicyclohexylamino group, pyrrolydyl group, piperidyl group, ditrifluoromethylamino group, phenylamino group, diphenylamino group, C₁-C₁₂ alkoxyphenylamino group, di(C₁-C₁₂ alkoxyphenyl)amino group, di(C₁-C₁₂ alkylphenyl)amino group, 1-naphthylamino group, 2-naphthylamino group, pentafluorophenylamino group, pyridylamino group, pyridazinylamino group, pyrimidylamino group, pyradylamino group, triazylamino group, phenyl-C₁-C₁₂ alkylamino group, C₁-C₁₂ alkoxyphenyl-C₁-C₁₂ alkylamino group, C₁-C₁₂ alkylphenyl-C₁-C₁₂ alkylamino group, di(C₁-C₁₂ alkoxyphenyl-C₁-C₁₂ alkyl)amino group, di(C₁-C₁₂ alkylphenyl-C₁-C₁₂ alkyl)amino group, 1-naphthyl-C₁-C₁₂ alkylamino group, 2-naphthyl-C₁-C₁₂ alkylamino group and the like.

The amide group has a carbon number of usually about 2 to 20, and specific examples thereof include a formamide group, acetamide group, propionamide group, butyramide group, benzamide group, trifluoroacetamide group, pentafluorobenzamide group, diformamide group, diacetamide group, dipropioamide group, dibutyroamide group, dibenzamide group, ditrifluoroacetamide group, dipentafluorobenzamide group and the like.

The acid imide group includes residues obtained by removing a hydrogen atom connected to a nitrogen atom of acid imide, and the carbon number is usually about 2 to 60, preferably 2 to 20. Specifically, the following groups are exemplified.

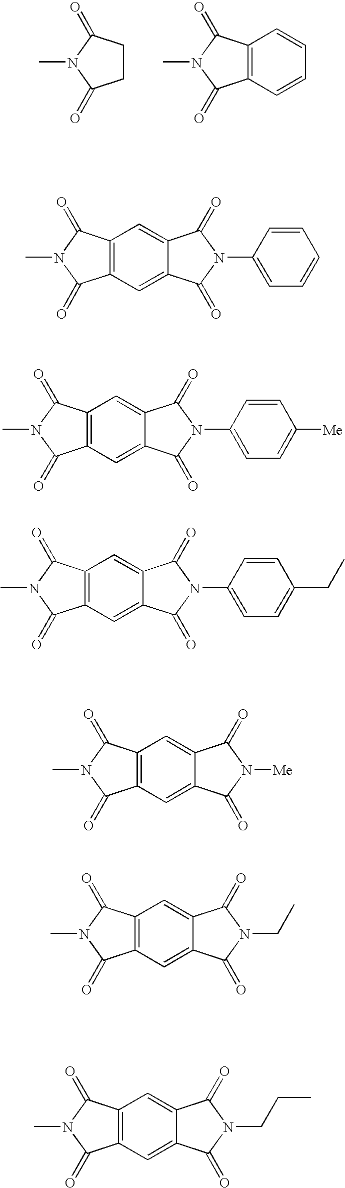

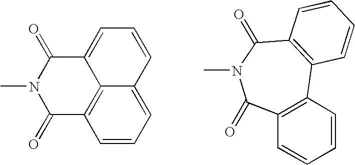

The acyloxy group has a carbon number of usually about 2 to 20, and specific examples thereof include an acetoxy group, propionyloxy group, butyryloxy group, isobutyryloxy group, pivaloyloxy group, benzoyloxy group, trifluoroacetyloxy group, pentafluorobenzoyloxy group and the like.

The mono-valent heterocyclic group means an atomic group remaining after removing one hydrogen atom from a heterocyclic compound, and the carbon number is usually about 2 to 60, and specific examples thereof include a thienyl group, C₁-C₁₂ alkylthienyl groups, pyrrolyl group, furyl group, pyridyl group, C₁-C₁₂ alkylpyridyl groups, imidazolyl group, pyrazolyl group, triazolyl group, oxazolyl group, thiazole group, thiadiazole group and the like.

The heteroaryloxy group (group of Q⁴-O—, Q⁴ represents a mono-valent heterocyclic group) has a carbon number of usually about 2 to 60, and specific examples thereof include a thienyloxy group, C₁-C₁₂ alkylthienyloxy groups, pyrrolyloxy group, furyloxy group, pyridyloxy group, C₁-C₁₂ alkylpyridyloxy groups, imidazolyloxy group, pyrazolyloxy group, triazolyloxy group, oxazolyloxy group, thiazoleoxy group, thiadiazoleoxy group and the like.

The heteroarylthio group (group of Q⁵-S—, Q⁵ represents a mono-valent heterocyclic group) has a carbon number of usually about 2 to 60, and specific examples thereof include a thienylmercapto group, C₁-C₁₂ alkylthienylmercapto groups, pyrrolylmercapto group, furylmercapto group, pyridylmercapto group, C₁-C₁₂ alkylpyridylmercapto groups, imidazolylmercapto group, pyrazolylmercapto group, triazolylmercapto group, oxazolylmercapto group, thiazolemercapto group, thiadiazolemercapto group and the like.

X represents an atom or atomic group forming a 5-membered or 6-membered ring together with four carbon atoms on two benzene rings in the formula (1a), and specific examples thereof include, but not limited to, the following moieties.

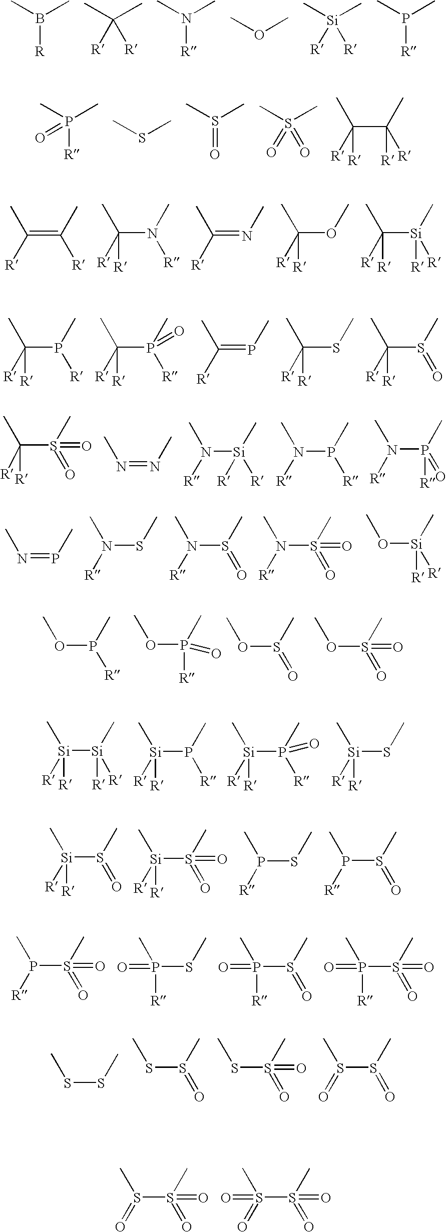

In the formulae, Rs represent each independently a halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyloxy group, substituted amino group, substituted silyloxy group, substituted silylthio group, substituted silylamino group, cyano group or mono-valent heterocyclic group. R′s represent each independently a hydrogen atom, halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyl group, acyloxy group, amide group, acid imide group, imine residue, substituted amino group, substituted silyl group, substituted silyloxy group, substituted silylthio group, substituted silylamino group, cyano group, nitro group or mono-valent heterocyclic group. R″s represent each independently a hydrogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyl group, substituted silyl group, substituted silyloxy group, substituted silylthio group, substituted silylamino group or mono-valent heterocyclic group.

Specific examples of the halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, substituted silyloxy group, substituted silylthio group, substituted silylamino group, substituted amino group, amide group, acid imide group, acyl group, acyloxy group and mono-valent heterocyclic group represented by R, R′ or R″ include those exemplified for Q and T in the formulae (1a) to (1d).

Of Xs, preferable are —O—, —S—, —Se—, —NR″—, —CR′R′— and —SiR′R′—, and more preferable are —O—, —S— and —CR′R′—.

The compound of the formula (1a) includes specifically the following compounds.

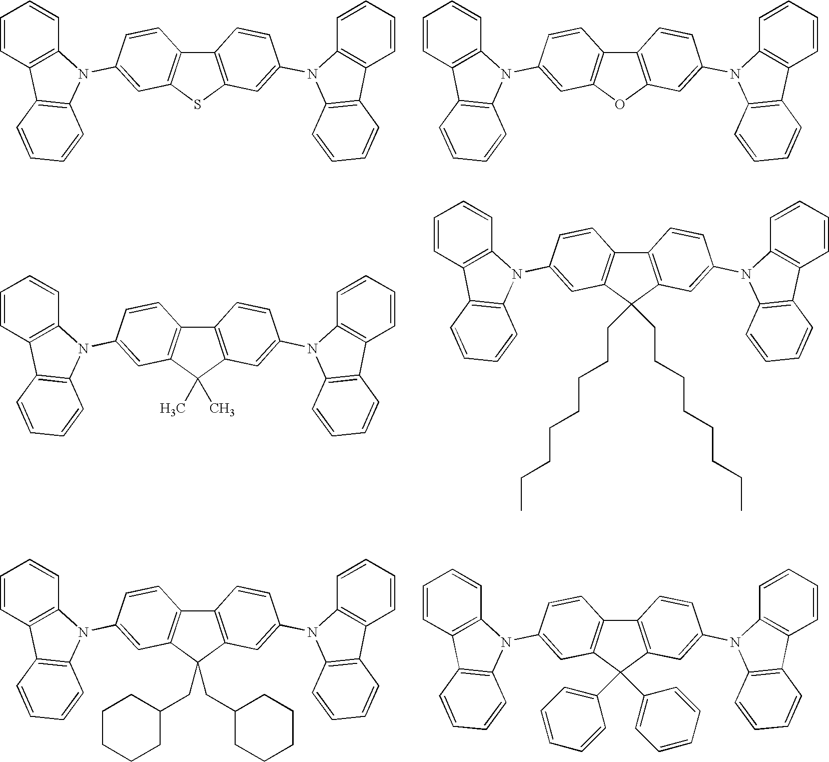

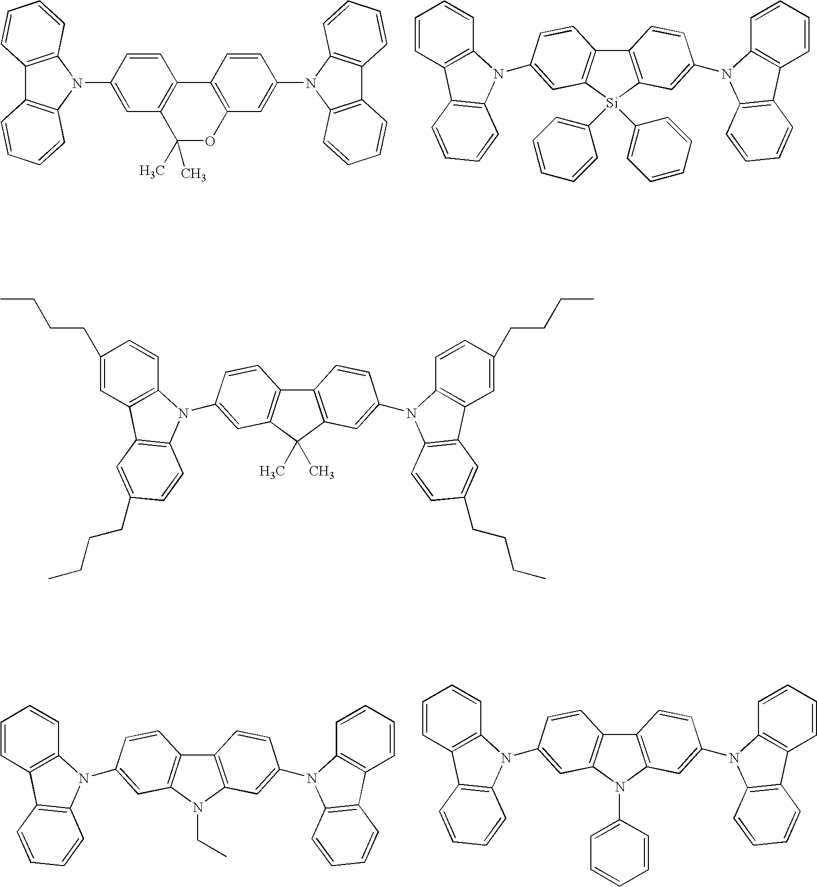

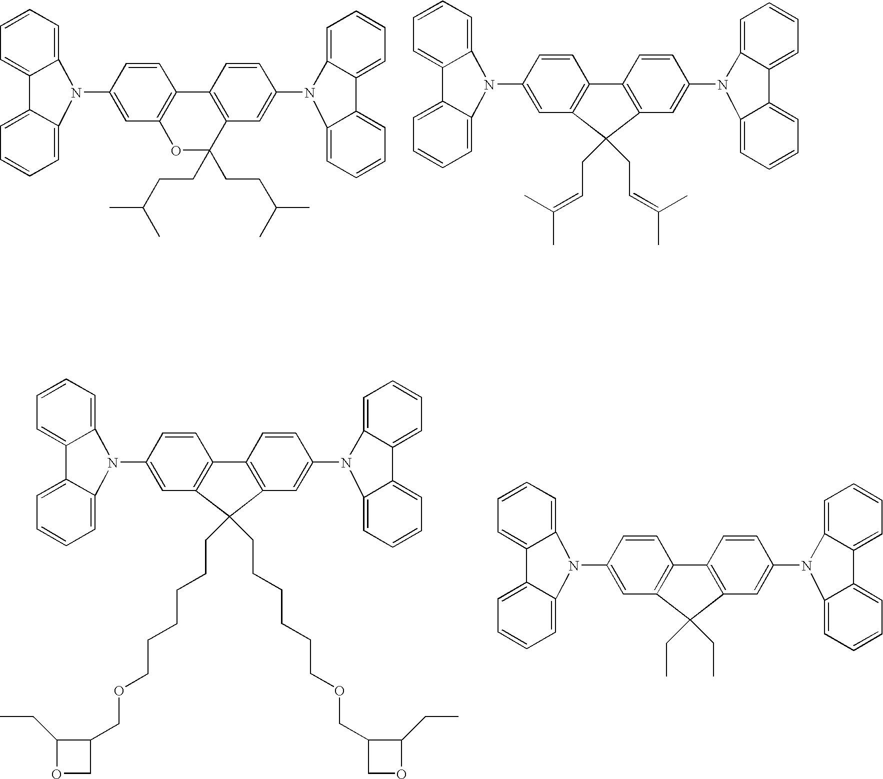

The compound of the formula (1b) includes specifically the following compounds.

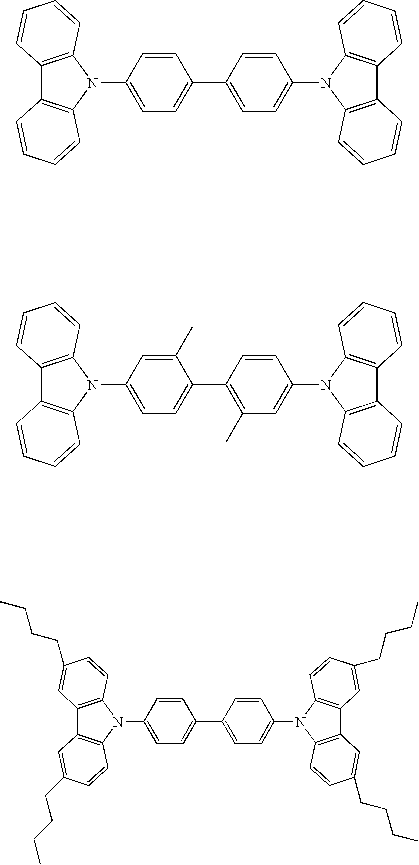

Of compounds of the formulae (1a) and (1b), the compound of the formula (1a) is more preferable from the standpoint of solubility in a solvent.

The compound of the formula (1c) includes specifically the following compounds.

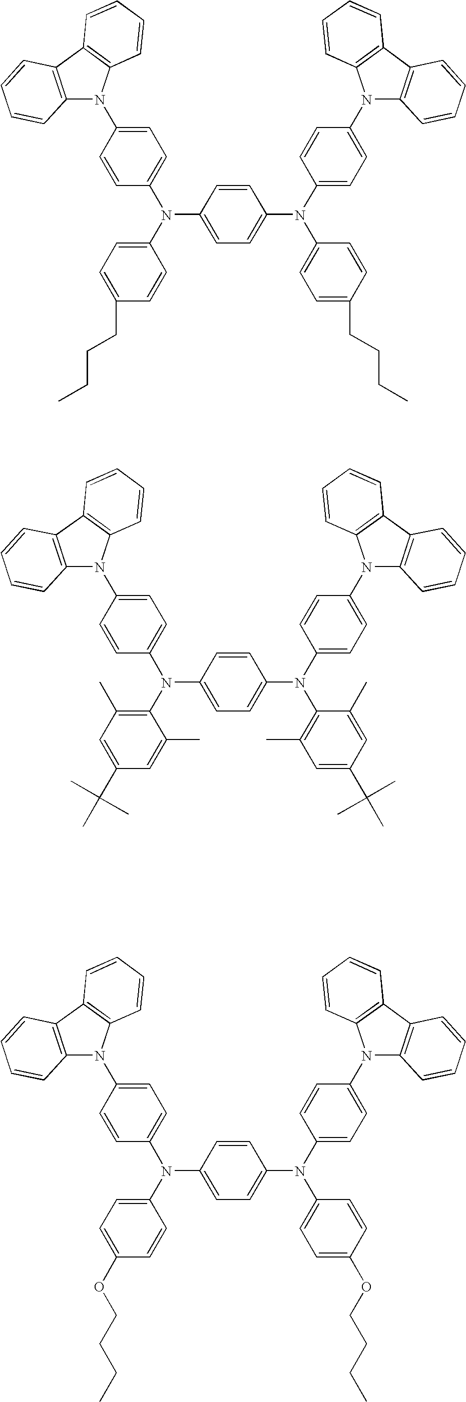

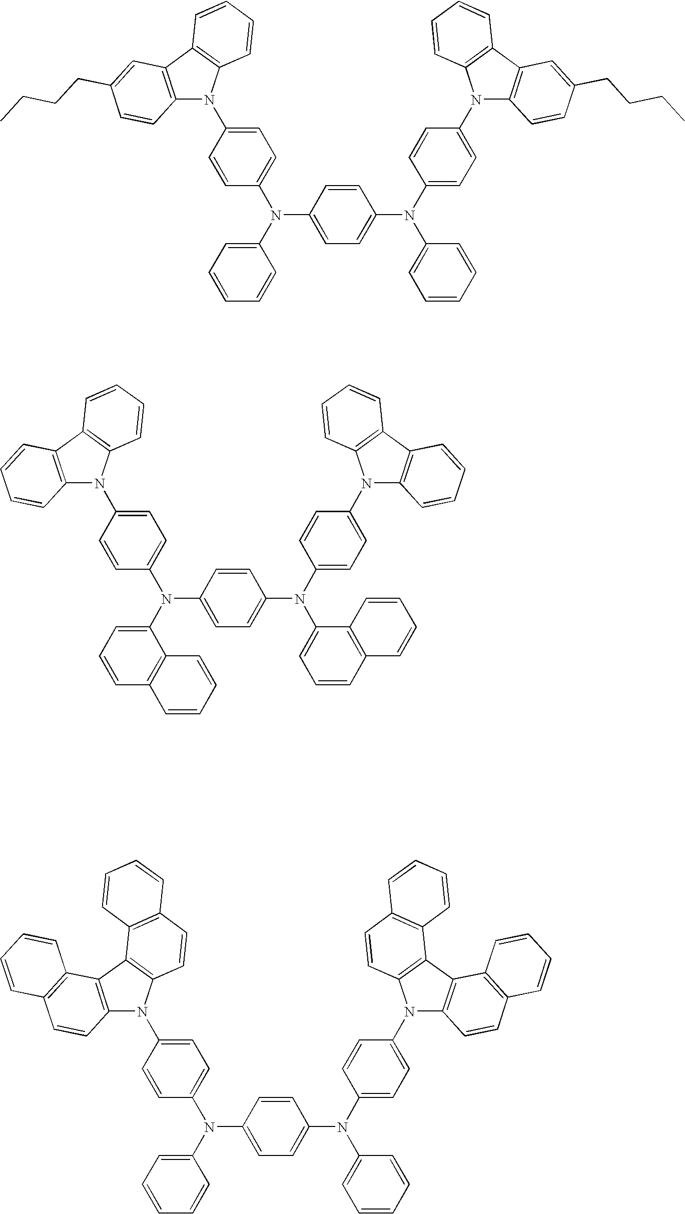

The compound of the formula (1d) includes specifically the following compounds.

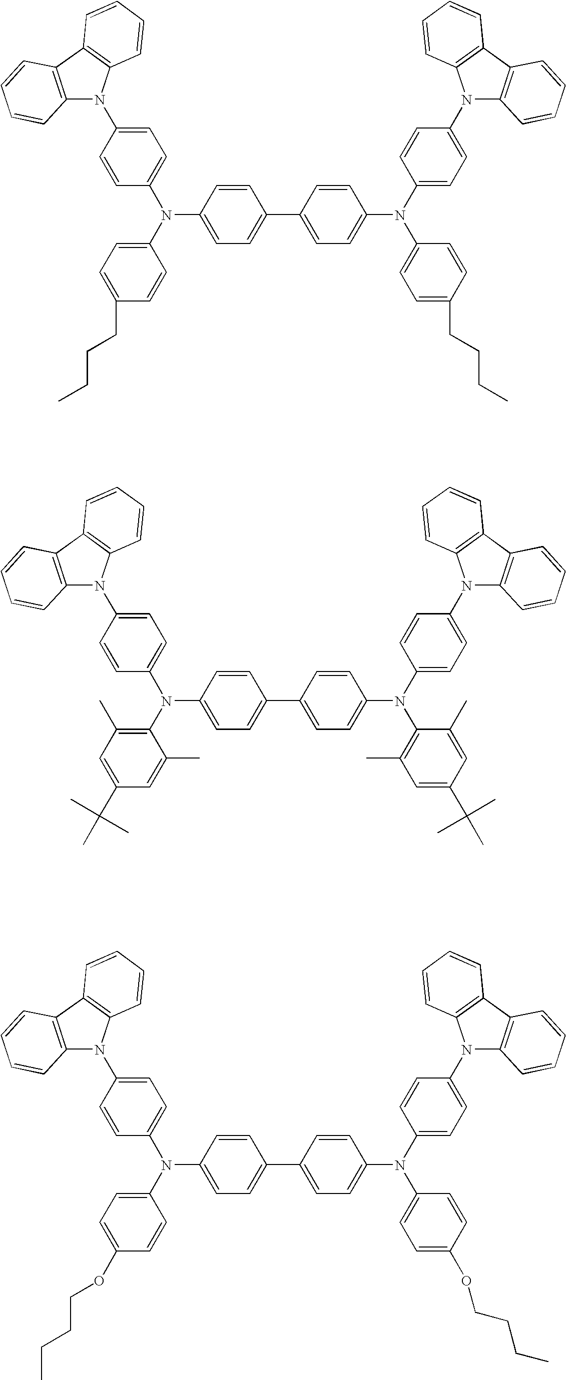

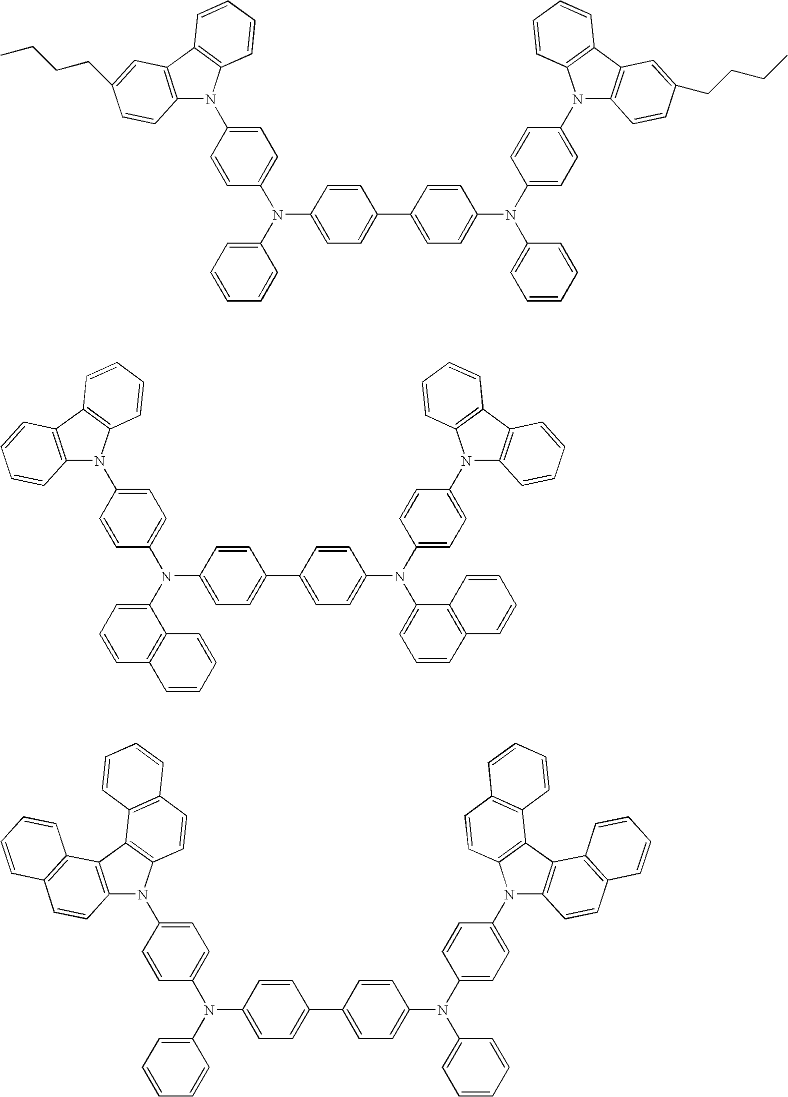

Among compounds (1a) to (1d), those in which T is selected from halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, substituted silyloxy group, substituted silylthio group, substituted silylamino group, substituted amino group, amide group, acid imide group, acyloxy group, mono-valent heterocyclic group, heteroaryloxy group, heteroarylthio group, cyano group and nitro group (other than hydrogen atom) are more preferable.

As methods for synthesizing compounds of the formulae (1a) to (1c) to be used in the present invention, there are exemplified cross coupling of a carbazole and a dibromodiamine derivative using a palladium catalyst, or coupling by the ullmann reaction and the like.

Next, the light emitting polymer to be used in the present invention will be described.

The light emitting polymer to be used in the present invention is not particularly restricted, and has a polystyrene-reduced number-average molecular weight of usually 10³ to 10⁸. The light emitting polymer to be used in the present invention may be a homo-polymer or copolymer.

Among light emitting polymers to be used in the present invention, conjugated polymer compounds are preferable. Here, the conjugated polymer compound means a polymer compound in which a delocalized p electron pair is present along the main chain skeleton of the polymer compound. Regarding this delocalized electron, unpaired electron or lone electron pair may participate in resonance instead of a double bond.

The light emitting polymer to be used in the present invention includes, for example, polyarylenes such as polyfluorene (e.g., Jpn. J. Appl. Phys.) vol. 30, L 1941 (1991)), polyparaphenylene (e.g., Adv. Mater.) vol. 4, 36 (1992)), polypyrrole, polypyridine, polyaniline, polythiophene and the like; polyarylenevinylenes such as polyparaphenylenevinylene, polythienylenevinylene and the like (e.g., WO98/27136 published specification); polyphenylene sulfide, polycarbazole and the like (general descriptions are found in, for example, “Advanced Materials Vol. 12, 1737-1750 (2000)”, and “Organic EL Display Technology, Monthly DISPLAY, December edition, Special issue, p 68-73”). Among them, polyarylene-based light emitting polymers are preferable.

As a repeating unit contained in the polyarylene-based light emitting polymer, mentioned are arylene groups and di-valent heterocyclic groups.

Here, the number of carbon atoms constituting a ring of an arylene group is usually about 6 to 60, and specific examples thereof include a phenylene group, biphenylene group, terphenylene group, naphthalenediyl group, anthracenediyl group, phenanthrenediyl group, pentalenediyl group, indenediyl group, heptalenediyl group, indacenediyl group, triphenylenediyl group, binaphthyldiyl group, phenylnaphthylenediyl group, stilbenediyl group, fluorenediyl group (e.g., in the case of A=-CR′R′— in the formula (2) below) and the like.

The number of carbon atoms constituting a ring of a di-valent heterocyclic group is usually about 3 to 60, and specific examples thereof include a pyridine-diyl group, diazaphenylene group, quinolinediyl group, quinoxalinediyl group, acridinediyl group, bipyridyldiyl group, phenanthrolinediyl group and a case in which A=—O—, —S—, —Se—, —NR″— or —SiR′R′— in the formula (2) below.

Further preferable is a case in which a repeating unit of the following formula (2) is contained.

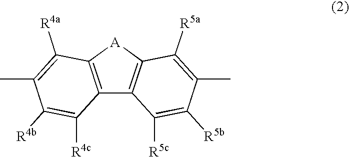

(wherein, A represents an atom or atomic group forming a 5-membered or 6-membered ring together with four carbon atoms on two benzene rings in the formula, R^{4a}, R^{4b}, R^{4c}, R^{5a}, R^{5b} and R^{5c} represent each independently a hydrogen atom, halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyl group, acyloxy group, amide group, acid imide group, imine residue, substituted amino group, substituted silyl group, substituted silyloxy group, substituted silylthio group, substituted silylamino group, cyano group, nitro group, mono-valent heterocyclic group, heteroaryloxy group, heteroarylthio group, alkyloxycarbonyl group, aryloxycarbonyl group, arylalkyloxycarbonyl group, heteroaryloxycarbonyl group or carboxyl group, and R^{4b} and R^{4c}, and R^{5b} and R^{5c} may together form a ring, respectively).

Specific examples of A include, but not limited to, those exemplified as specific examples of X in the formula (1a).

Of As, preferable are —O—, —S—, —Se—, —NR″—, —CR′R′— and —SiR′R′—, and more preferable are —O—, —S— and —CR′R′—.

The halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyloxy group, amide group, acid imide group, substituted amino group, substituted silyloxy group, substituted silylthio group, substituted silylamino group, mono-valent heterocyclic group, heteroaryloxy group, heteroarylthio group and carboxyl group represented by R^{4a}, R^{4b}, R^{4c}, R^{5a}, R^{5b} and R^{5c} are the same as described above.

The imine residue includes residues obtained by removing one hydrogen atom from imine compounds (meaning organic compounds having —N═C— in the molecule. Examples thereof include aldimines, ketimines and compounds obtained by substituting a hydrogen atom on N of these compounds by an alkyl group and the like), and the carbon number is usually about 2 to 20, and specifically, the following groups and the like are exemplified.

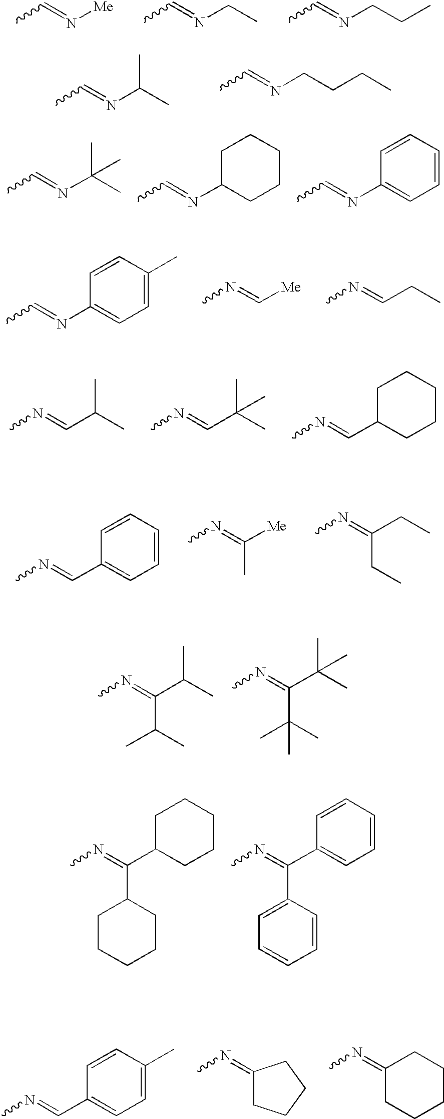

The acyl group has a carbon number of usually about 2 to 20, and specific examples thereof include an acetyl group, propionyl group, butyryl group, isobutyryl group, pivaloyl group, benzoyl group, trifluoroacetyl group, pentafluorobenzoyl group and the like.

The substituted silyl group includes silyl groups substituted with 1, 2 or 3 groups selected from alkyl groups, aryl groups, arylalkyl groups and mono-valent heterocyclic groups. The substituted silyl group has a carbon number of usually 1 to about 60, and specific examples thereof include a trimethylsilyloxy group, triethylsilyloxy group, tripropylsilyloxy group, tri-i-propylsilyloxy group, dimethyl-1-propylsilyl group, diethyl-1-propylsilyl group, t-butylsilyldimethylsilyl group, pentyldimethylsilyl group, hexyldimethylsilyl group, heptyldimethylsilyl group, octyldimethylsilyl group, 2-ethylhexyl-dimethylsilyl group, nonyldimethylsilyl group, decyldimethylsilyl group, 3,7-dimethyloctyl-dimethylsilyl group, lauryldimethylsilyl group, phenyl-C₁-C₁₂-alkylsilyl groups, C₁-C₁₂-alkoxyphenyl-C₁-C₁₂-alkylsilyl groups, C₁-C₁₂-alkylphenyl-C₁-C₁₂-alkylsilyl groups, 1-naphthyl-C₁-C₁₂-alkylsilyl groups, 2-naphthyl-C₁-C₁₂-alkylsilyl groups, phenyl-C₁-C₁₂-alkyldimethylsilyl groups, triphenylsilyl group, tri-p-xylylsilyl group, tribenzylsilyl group, diphenylmethylsilyl group, t-butyldiphenylsilyl group, dimethylphenylsilyl group, trimethoxysilyl group, triethoxysilyl group, tripropyloxysilyl group, tri-i-propylsilyl group, dimethyl-i-propylsilyl group, methyldimethoxysilyl group, ethyldimethoxysilyl group and the like.

The alkyloxy group in the alkyloxycarbonyl group has a carbon number of usually about 2 to 20, and specific examples thereof include an acetoxy group, propionyloxy group, butyryloxy group, isobutyryloxy group, pivaloyloxy group, benzoyloxy group, trifluoroacetyloxy group, pentafluorobenzoyloxy group and the like.

The aryloxy group in the aryloxycarbonyl group has a carbon number of usually about 6 to 60, and specific examples thereof include a phenoxy group, C₁-C₁₂ alkoxyphenoxy groups, C₁-C₁₂ alkylphenoxy groups, 1-naphthyloxy group, 2-naphthyloxy group, pentafluorophenyloxy group and the like, and preferable are C₁-C₁₂ alkoxyphenoxy groups and C₁-C₁₂ alkylphenoxy groups.

The arylalkyl group in the arylalkyloxycarbonyl group has a carbon number of usually about 7 to 60, and specific examples thereof include phenyl-C₁-C₁₂ alkyl groups such as a phenylmethyl group, phenylethyl group, phenylbutyl group, phenylpentyl group, phenylhexyl group, phenylheptyl group, phenyloctyl group and the like; C₁-C₁₂ alkoxyphenyl-C₁-C₁₂ alkyl groups, C₁-C₁₂ alkylphenyl-C₁-C₁₂ alkyl groups, 1-naphthyl-C₁-C₁₂ alkyl groups, 2-naphthyl-C₁-C₁₂ alkyl groups and the like, and preferable are C₁-C₁₂ alkoxyphenyl-C₁-C₁₂ alkyl groups and C₁-C₁₂ alkylphenyl-C₁-C₁₂ alkyl groups.

The heteroaryloxy group (group of Q⁶—O—, Q⁶ represents a mono-valent heterocyclic group) in the heteroaryloxycarbonyl group has a carbon number of usually about 2 to 60, and specific examples thereof include a thienyloxy group, C₁-C₁₂ alkylthienyloxy groups, pyrrolyloxy group, furyloxy group, pyridyloxy group, C₁-C₁₂ alkylpyridyloxy groups, imidazolyloxy group, pyrazolyloxy group, triazolyloxy group, oxazolyloxy group, thiazoleoxy group, thiadiazoleoxy group and the like. Preferable as Q⁶ are mono-valent aromatic heterocyclic groups.

As the repeating unit of the formula (2), the following structures are exemplified.

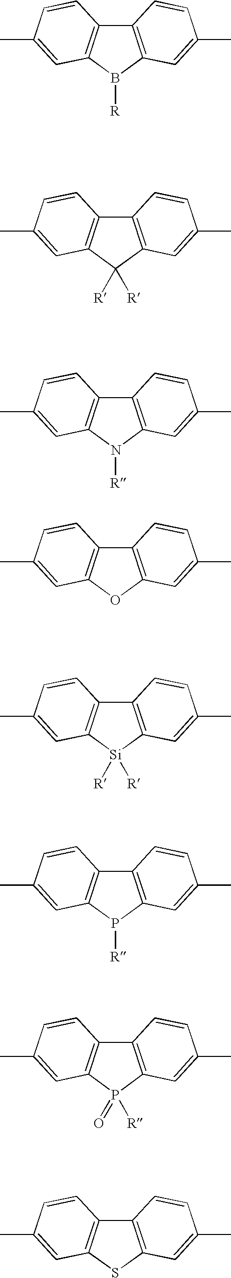

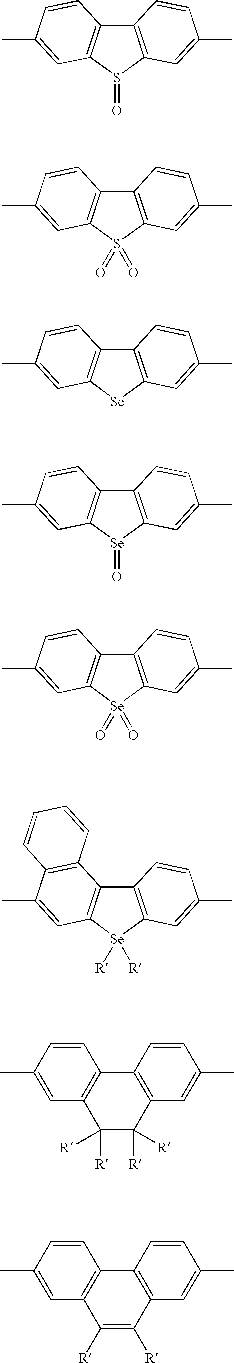

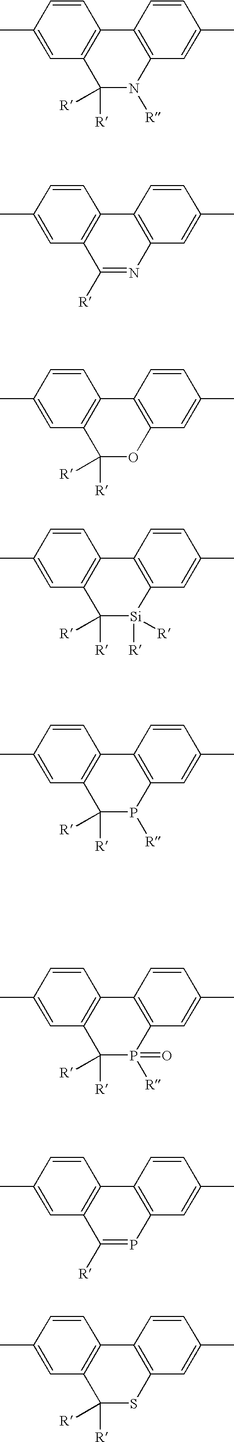

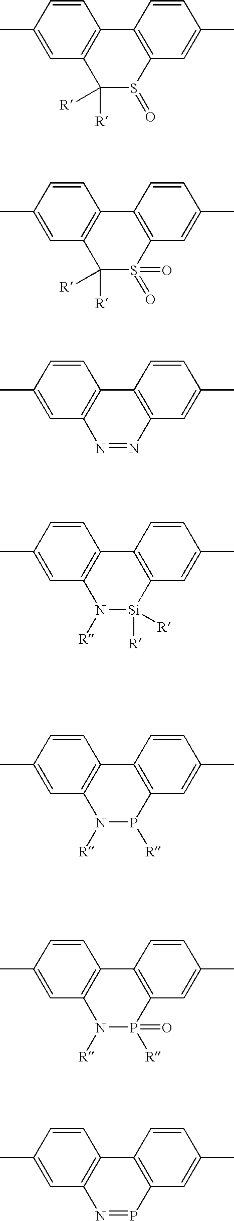

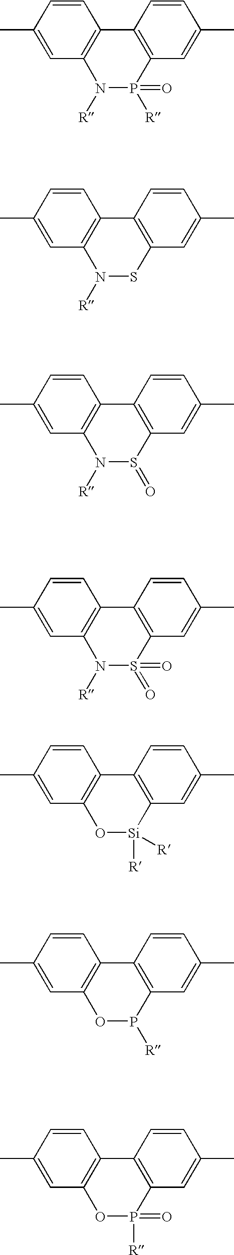

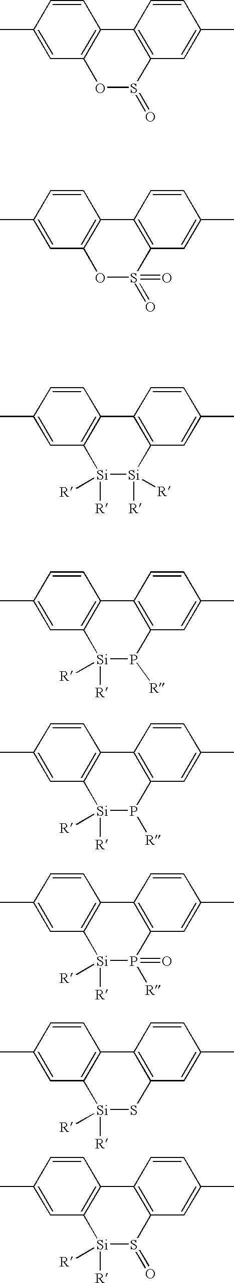

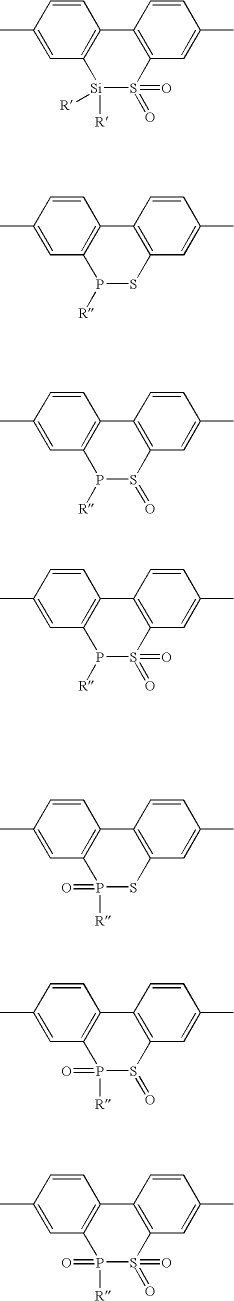

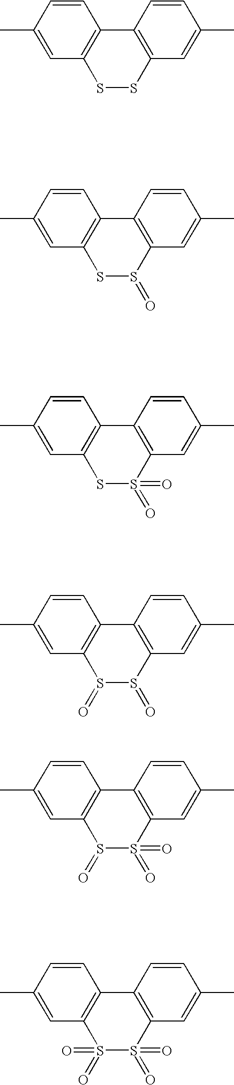

In the formulae, a hydrogen atom on a benzene ring may be substituted by a halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyl group, acyloxy group, amide group, acid imide group, imine residue, substituted amino group, substituted silyl group, substituted silyloxy group, substituted silylthio group, substituted silylamino group, cyano group, nitro group or mono-valent heterocyclic group.

The light emitting polymer to be used in the present invention may contain, for example, a repeating unit derived from an aromatic amine, in addition to an arylene group and a di-valent heterocyclic group. In this case, hole injectability and transportability can be imparted.

In this case, the molar ratio of repeating units composed of an arylene group and a di-valent heterocyclic group to repeating units derived from an aromatic amine is usually in the range of 99:1 to 20:80.

As the repeating unit derived from an aromatic amine, preferable are repeating units of the following formula (3).

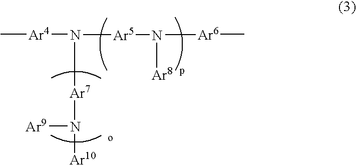

In the formula, Ar⁴, Ar⁵, Ar⁶ and Ar⁷ represent each independently an arylene group or di-valent heterocyclic group. Ar⁸, Ar⁹ and Ar¹⁰ represent each independently an aryl group or mono-valent heterocyclic group. o and p represent each independently 0 or 1, and 0=o+p=2.

Here, specific examples of an arylene group and a di-valent heterocyclic group are the same as specific examples of the repeating unit contained in polyarylene-based light emitting polymers, and specific examples of an aryl group and a mono-valent heterocyclic group are the same as specific examples of the above-described formulae (1a) to (1d).

As specific examples of the repeating unit of the formula (3), the following repeating units are mentioned.

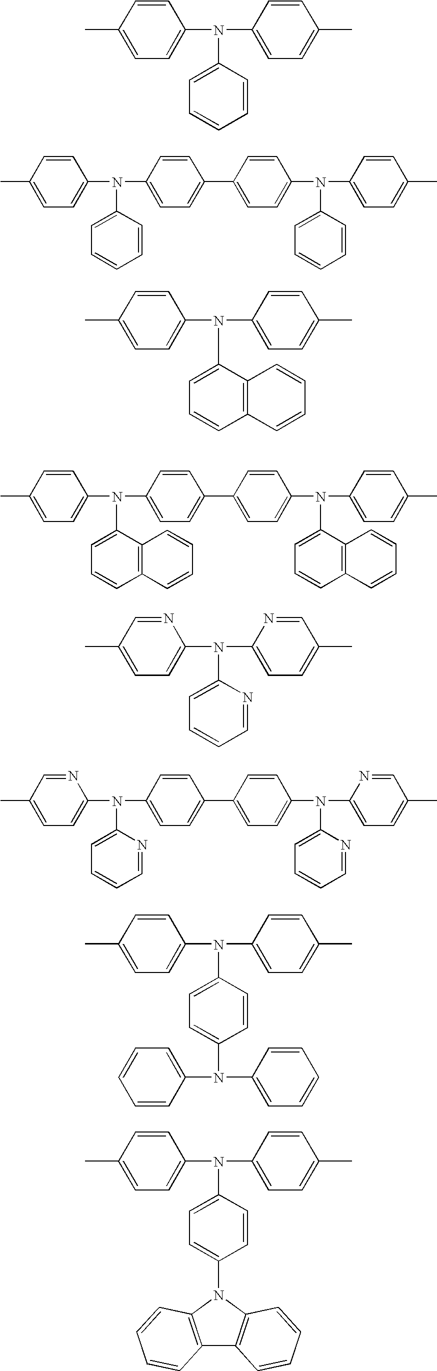

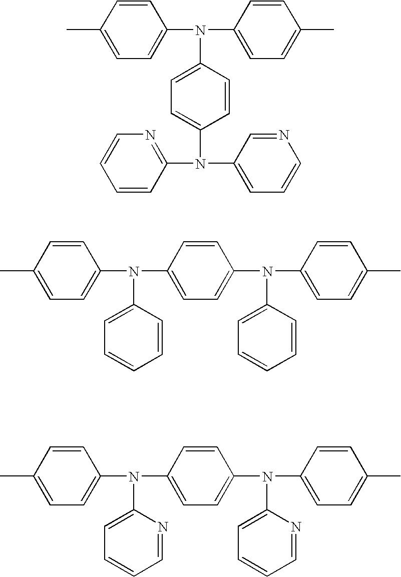

In the formulae, a hydrogen atom on an aromatic ring may be substituted by a substituent selected from halogen atom's alkyl groups, alkyloxy groups, alkylthio groups, aryl groups, aryloxy groups, arylthio groups, arylalkyl groups, arylalkyloxy groups, arylalkylthio groups, alkenyl groups, alkynyl groups, arylalkenyl groups, arylalkynyl groups, acyl groups, acyloxy groups, amide groups, acid imide groups, imine residues, substituted amino groups, substituted silyl groups, substituted silyloxy groups, substituted silylthio groups, substituted silylamino groups, cyano group, nitro group, mono-valent heterocyclic groups, heteroaryloxy groups, heteroarylthio groups, alkyloxycarbonyl groups, aryloxycarbonyl groups, arylalkyloxycarbonyl groups, heteroaryloxycarbonyl groups and carboxyl group.

Among repeating units of the above-described formula (3), repeating units of the following formula (4) are particularly preferable.

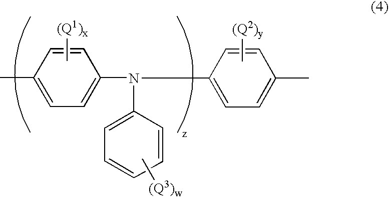

In the formula, Q¹, Q² and Q³ represent each independently a halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyl group, acyloxy group, amide group, acid imide group, imine residue, substituted amino group, substituted silyl group, substituted silyloxy group, substituted silylthio group, substituted silylamino group, cyano group, nitro group, mono-valent heterocyclic group, heteroaryloxy group, heteroarylthio group, alkyloxycarbonyl group, aryloxycarbonyl group, arylalkyloxycarbonyl group, heteroaryloxycarbonyl group or carboxyl group. x and y represent each independently an integer of 0 to 4. z represents an integer of 0 to 2. w represents an integer of 0 to 5.

The light emitting polymer to be used in the present invention may be a random, block or graft copolymer, alternatively, a polymer having a structure which is intermediate between them, for example, a random copolymer partaking a blocking property. Random copolymers partaking a blocking property, and block or graft copolymers are more preferable than complete random copolymers, from the standpoint of obtaining a light emitting polymer of high quantum yield of emission. Those having branching in the main chain and having three or more end parts, and dendrimers are also included.

An end group of the light emitting polymer to be used in the present invention may be protected by a stable group since when a polymerization active group remains intact, there is s possibility of decrease in light emitting properties and durability when made into an element. Those having a conjugated bond consecutive to a conjugated structure of the main chain are preferable, and for example, structures connecting to an aryl group or heterocyclic group via a carbon-carbon bond are exemplified. Specifically, substituents described in (chemical formula 10) in Japanese Patent Application Laid-Open (JP-A) No. 9-45478, and the like are exemplified.

The light emitting polymer to be used in the present invention has preferably a number-average molecular weight reduced by polystyrene of about 10³ to 10⁸, further preferably a number-average molecular weight reduced by polystyrene of about 10⁴ to 10⁶.

As the light emitting polymer, those showing emission at solid condition are preferably used since light emission from a thin membrane is utilized.

As the method for synthesizing a light emitting polymer to be used in the present invention, for example, a method for polymerizing from the corresponding monomers by the Suzuki coupling reaction, a method of polymerization by the Grignard reaction, a method of polymerization using a Ni(0) catalyst, a method of polymerization using an oxidizer such as FeCl₃ and the like, a method of electrochemical oxidation polymerization, a method by decomposition of an intermediate polymer having a suitable releasing group, and the like are exemplified. Of them, a method of polymerization by the Suzuki coupling reaction, a method of polymerization by the Grignard reaction and a method of polymerization by a Ni(0) catalyst are preferable since reaction control is easy.

When the light emitting polymer is used as an emitting material for a polymer LED, its purity influences the emitting property, thus, it is preferable that a monomer before polymerization is purified by a method such as distillation, sublimation purification, re-crystallization and the like before polymerization, and it is preferable that after synthesis, a purification treatment such as re-deposition purification, chromatography fractionation and the like is performed.

The light emitting polymer composition of the present invention is characterized in that it contains a light emitting polymer and a compound selected from the formulae (1a) to (1d), and the content of the compound selected from the formulae (1a) to (1d) is usually about 0.1 to 10000 parts by weight, preferably 1 to 1000 parts by weight, more preferably 5 to 500 parts by weight, further preferably 10 to 100 parts by weight, based on 100 parts by weight of the light emitting polymer.

The light emitting polymer solution composition of the present invention is characterized in that it contains a light emitting polymer, a compound selected from the formulae (1a) to (1d) and a solvent. Using this solution composition, alight emitting layer can be formed by an application method. The light emitting layer produced using this solution composition usually contains a light emitting polymer composition of the present invention.

As the solvent, chloroform, methylene chloride, dichloroethane, tetrahydrofuran, toluene, xylene, mesitylene, tetralin, decalin, n-butylbenzene and the like are exemplified. Depending on the structure and molecular weight of a light emitting polymer, the light emitting polymer can be dissolved in an amount of usually 0.1 wt % or more in these solvents.

The amount of the solvent is usually about 1000 to 100000 parts by weight based on 100 parts by weight of a light emitting polymer.

The light emitting polymer composition of the present invention may contain two or more light emitting polymers, and may also contain two or more compounds of the formulae (1a) to (1d). Further, the composition of the present invention may also contain a coloring matter, charge transport material and the like, if required.

The polymer LED of the present invention is characterized in that it has a light emitting layer between electrodes composed of an anode and a cathode and the light emitting layer contains a light emitting polymer composition of the present invention. The polymer LED of the present invention is characterized in that it has a light emitting layer between electrodes composed of an anode and a cathode and the light emitting layer is formed using a solution composition of the present invention.

As the polymer LED of the present invention, mentioned are a polymer LED having an electron transport layer provided between a cathode and a light emitting layer, a polymer LED having a hole transport layer provided between an anode and a light emitting layer, a polymer LED having an electron transport layer provided between a cathode and a light emitting layer, and having a hole transport layer provided between an anode and a light emitting layer.

For example, the following structures a) to d) are specifically exemplified.

a) anode/light emitting layer/cathode

b) anode/hole transport layer/light emitting layer/cathode

c) anode/light emitting layer/electron transport layer/cathode

d) anode/hole transport layer/light emitting layer/electron transport layer/cathode

(wherein, / shows adjacent lamination of layers, applicable in the followings.)

Here, the light emitting layer is a layer having a function of light emission, the hole transport layer is a layer having a function of transporting holes, and the electron transport layer is a layer having a function of transporting electrons. The electron transport layer and the hole transport layer are generically called charge transport layer. Two or more light emitting layers, two or more hole transport layers and two or more electron transport layers may be used each independently.

Of charge transport layers provided adjacent to an electrode, those having a function of improving efficiency of charge injection from an electrode and having an effect of lowering the driving voltage of a device are sometimes referred to particularly as charge injection layer (hole injection layer, electron injection layer) in general.

Further, for enhancement of close adherence with an electrode and improvement of charge injection from an electrode, the above-described charge injection layer or an insulation layer having a thickness of 2 nm or less may be provided, and for enhancement of close adherence of an interface and prevention of mixing and the like, a thin buffer layer may be inserted at an interface of charge transport layers and light emitting layers.

The order and number of layers to be laminated, and the thickness of each layer can be appropriately selected taking emitting efficiency and device life into consideration.

In the present invention, mentioned as the polymer LED having a charge injection layer (electron injection layer, hole injection layer) are a polymer LED having a charge injection layer provided adjacent to a cathode and a polymer LED having a charge injection layer adjacent to an anode.

For example, the following structures e) to p) are specifically mentioned.

e) anode/charge injection layer/light emitting layer/cathode

f) anode/light emitting layer/charge injection layer/cathode

g) anode/charge injection layer/light emitting layer/charge injection layer/cathode

h) anode/charge injection layer/hole transport layer/light emitting layer/cathode

i) anode/hole transport layer/light emitting layer/charge injection layer/cathode

j) anode/charge injection layer/hole transport layer/light emitting layer/charge injection layer/cathode

k) anode/charge injection layer/light emitting layer/electron transport layer/cathode

l) anode/light emitting layer/electron transport layer/charge injection layer/cathode

m) anode/charge injection layer/light emitting layer/electron transport layer/charge injection layer/cathode

n) anode/charge injection layer/hole transport layer/light emitting layer/electron transport layer/cathode

o) anode/hole transport layer/light emitting layer/electron transport layer/charge injection layer/cathode

p) anode/charge injection layer/hole transport layer/light emitting layer/electron transport layer/charge injection layer/cathode

As specific examples of the charge injection layer, exemplified are a layer containing a conductive polymer, a layer provided between an anode and a hole transport layer and having an ionization potential of a value between that of an anode material and that of a hole transport material contained in the hole transport layer, a layer provided between a cathode and an electron transport layer and having an electron affinity of a value between that of a cathode material and that of an electron transport material contained in the electron transport layer, and the like.

When the above-described charge injection layer is a layer containing a conductive polymer, the electric conductivity of the conductive polymer is preferably 10⁻⁵ S/cm or more and 10³ S/cm or less, and for lowering the leak current between light emitting pixels, more preferably 10⁻⁵ S/cm or more and 10² S/cm or less, and further preferably 10⁻⁵ S/cm or more and 10¹ S/cm or less.

Usually, a suitable amount of ions are doped into the conductive polymer for the electric conductivity of the conductive polymer to be 10⁻⁵ S/cm or more and 103 S/cm or less.

The kind of the ion to be doped is an anion in the case of a hole injection layer, and a cation in the case of an electron injection layer. Examples of the anion include a polystyrenesulfonate ion, alkylbenzenesulfonate ion, camphorsulfonate ion and the like, and examples of the cation include a lithium ion, sodium ion, potassium ion, tetrabutylammonium ion and the like.

The thickness of the charge injection layer is, for example, 1 nm to 100 nm, and preferably 2 nm to 50 nm.

The material used in the charge injection layer may be appropriately selected in view of a correlation with materials of electrodes and adjacent layers, and exemplified are polyaniline and derivatives thereof, polythiophene and derivatives thereof, polypyrrole and derivatives thereof, polyphenylenevinylene and derivatives thereof, polythienylenevinylene and derivatives thereof, polyquinoline and derivatives thereof, polyquinoxaline and derivatives thereof, conductive polymers such as a polymer containing an aromatic amine structure in the main chain or side chain and the like, metal phthalocyanines (copper phthalocyanine and the like), carbon, and the like.

The insulation layer having a thickness of 2 nm or less has a function of facilitating charge injection. As the material of the insulation layer, metal fluorides, metal oxides, organic insulation materials and the like are mentioned. As the polymer LED having an insulation layer having a thickness of 2 nm or less, there are a polymer LED having an insulation layer having a thickness of 2 nm or less provided adjacent to a cathode, and a polymer LED having an insulation layer having a thickness of 2 nm or less provided adjacent to an anode.

Specifically, the following structures q) to ab) are mentioned, for example.

q) anode/insulation layer of 2 nm or less thickness/light emitting layer/cathode

r) anode/light emitting layer/insulation layer of 2 nm or less thickness/cathode

s) anode/insulation layer of 2 nm or less thickness/light emitting layer/insulation layer of 2 nm or less thickness/cathode

t) anode/insulation layer of 2 nm or less thickness/hole transport layer/light emitting layer/cathode

u) anode/hole transport layer/light emitting layer/insulation layer of 2 nm or less thickness/cathode

v) anode/insulation layer of 2 nm or less thickness/hole transport layer/light emitting layer/insulation layer of 2 nm or less thickness cathode

w) anode/insulation layer of 2 nm or less thickness/light emitting layer/electron transport layer/cathode

x) anode/light emitting layer/electron transport layer/insulation layer of 2 nm or less thickness/cathode

y) anode/insulation layer of 2 nm or less thickness/light emitting layer/electron transport layer/insulation layer of 2 nm or less thickness/cathode

z) anode/insulation layer of 2 nm or less thickness/hole transport layer/light emitting layer/electron transport layer/cathode

aa) anode/hole transport layer/light emitting layer/electron transport layer/insulation layer of 2 nm or less thickness/cathode

bb) anode/insulation layer of 2 nm or less thickness/hole transport layer/light emitting layer/electron transport layer/insulation layer of 2 nm or less thickness/cathode

When, for example, the light emitting layer is formed from a solution using a light emitting polymer solution composition of the present invention, it is sufficient that this solution is applied before removal of a solvent by drying, and also when a charge transport material or a light emitting material is mixed, the same method is applicable and very advantageous from the standpoint of production. As the method of film formation from a solution, application methods such as a spin coat method, casting method, micro gravure coat method, gravure coat method, bar coat method, roll coat method, wire bar coat method, dip coat method, spray coat method, screen printing method, flexographic printing method, offset printing method, inkjet printing method and the like can be used.

Regarding the thickness of a light emitting layer, the optimum value varies depending on the material to be used and may be advantageously selected so that the driving voltage and emitting efficiency manifest suitable values, and it is, for example, from 1 nm to 1 μm, preferably 2 nm to 500 nm, further preferably 5 nm to 200 nm.

In the polymer LED of the present invention, an emitting material other than the above-described light emitting polymer may be mixed and used in a light emitting layer. A light emitting layer containing an emitting material other than the above-described light emitting polymer may also be laminated with a light emitting layer containing the above-described light emitting polymer.

As the emitting material, known materials can be used. In the case of compounds of low molecular weight, for example, naphthalene derivatives, anthracene or derivatives thereof, perylene or derivatives thereof, coloring matters such as polymethines, xanthenes, coumarins, cyanines and the like, metal complexes of 8-hydroxyquinoline or derivatives thereof, aromatic amines, tetraphenylcyclopentadiene or derivatives thereof, tetraphenylbutadiene or derivatives thereof, and the like can be used.

Specifically, known compounds such as those described, for example, in JP-A Nos. 57-51781 and 59-194393, and the like can be used.

Mentioned as triplet light emitting complexes are, for example, Ir(ppy)3 containing iridium as a center metal, Btp₂Ir(acac), PtOEP containing platinum as a center metal, Eu(TTA)3phen containing europium as a center metal, and the like.

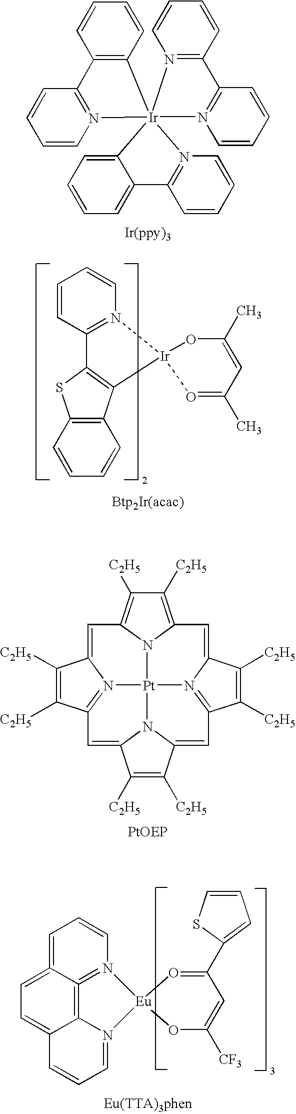

Specific examples of the triplet light emitting complex are described in, for example, Nature, (1998), 395, 151, Appl. Phys. Lett. (1999), 75(1), 4, Proc. SPIE-Int. Soc. Opt. Eng. (2001), 4105 (Organic Light-Emitting Materials and Devices IV), 119, J. Am. Chem. Soc., (2001), 123, 4304, Appl. Phys. Lett., (1997), 71(18), 2596, Syn. Met., (1998), 94(1), 103, Syn. Met., (1999), 99(2), 1361, Adv. Mater., (1999), 11(10), 852, Jpn. J. Appl. Phys., 34, 1883 (1995), and the like.

When the polymer LED of the present invention has a hole transport layer, exemplified as the hole transport material to be used are polyvinylcarbazole or derivatives thereof, polysilane or derivatives thereof, polysiloxane derivatives having an aromatic amine in the side chain or main chain, pyrazoline derivatives, arylamine derivatives, stillbene derivatives, triphenyldiamine derivatives, polyaniline or derivatives thereof, polythiophene or derivatives thereof, polypyrrole or derivatives thereof, poly(p-phenylenevinylene) or derivatives thereof, poly(2,5-thienylenevinylene) or derivatives thereof, and the like.

Specifically, exemplified as the hole transport material are those described in JP-A Nos. 63-70257, 63-175860, 2-135359, 2-135361, 2-209988, 3-37992 and 3-152184, and the like.

Among them, preferable as the hole transport material used in the hole transport layer are polymer hole transport materials such as polyvinylcarbazole or derivatives thereof, polysilane or derivatives thereof, polysiloxane derivatives having an aromatic amine compound group in the side chain or main chain, polyaniline or derivatives thereof, polythiophene or derivatives thereof, poly(p-phenylenevinylene) or derivatives thereof, poly(2,5-thienylenevinylene) or derivatives thereof, and the like, and further preferable are polyvinylcarbazole or derivatives thereof, polysilane or derivatives thereof and polysiloxane derivatives having an aromatic amine in the side chain or main chain. In the case of a hole transport material of low molecular weight, the material is preferably dispersed in a polymer binder.

The polyvinylcarbazole or derivatives thereof are obtained, for example, by cation polymerization or radical polymerization from vinyl monomers.

As the polysilane or derivatives thereof, exemplified are compounds described in Chem. Rev., vol. 89, 1359 (1989), GB 2300196, and the like. Synthesis methods described in these literatures can be used, and in particular, the Kipping method is suitably used.

As the polysiloxane or derivatives thereof, those having a structure of the above-described hole transport material of low molecular weight in the side chain or main chain are suitably used since the siloxane skeleton structure has little hole transportability. Particularly, those having a hole transportable aromatic amine in the side chain or main chain are exemplified.

The method for forming the hole transport layer is not particularly restricted, and in the case of the hole transport material of low molecular weight, exemplified is a method for film formation from a mixed solution with a polymer binder. In the case of the hole transport material of high molecular weight, exemplified is a method for film formation from a solution.

The solvent to be used for film formation from a solution is not particularly restricted providing it dissolves a hole transport material. Exemplified as the solvent are chlorine-based solvents such as chloroform, methylene chloride, dichloroethane and the like, ether-based solvents such as tetrahydrofuran and the like, aromatic hydrocarbon-based solvents such as toluene, xylene and the like, ketone-based solvents such as acetone, methyl ethyl ketone and the like, and ester-based solvents such as ethyl acetate, butyl acetate, ethyl cellosolve acetate and the like.

As the method of film formation from a solution, application methods such as a spin coat method from a solution, casting method, micro gravure coat method, gravure coat method, bar coat method, roll coat method, wire bar coat method, dip coat method, spray coat method, screen printing method, flexographic printing method, offset printing method, inkjet printing method and the like can be used.

As the polymer binder to be mixed, those not extremely disturb charge transport are preferable, and those manifesting no strong absorption for visible light are suitably used. As the polymer binder, polycarbonate, polyacrylate, polymethyl acrylate, polymethyl methacrylate, polystyrene, polyvinyl chloride, polysiloxane and the like are exemplified.

Regarding the thickness of a hole transport layer, the optimum value varies depending on the material to be used and may be advantageously selected so that the driving voltage and emitting efficiency manifest suitable values, and at least thicknesses causing no generation of pin holes are necessary, and when the thickness is too large, the driving voltage of a device increases undesirably. Therefore, the thickness of the hole transport layer is, for example, from 1 nm to 1 μm, preferably 2 nm to 500 nm, further preferably 5 nm to 200 nm.

In the polymer LED composition of the present invention, further higher efficiency can be obtained by combining with a hole transport layer made of a polyamine having a repeating unit derived particularly from an aromatic amine. As the polyamine, those containing repeating unit of the formula (3) are preferable, and further preferably, those containing repeating unit of the formula (4) are advantageous.

When the polymer LED of the present invention has an electron transport layer, known materials can be used as the electron transport material, and exemplified are oxadiazole derivatives, anthraquinodimethane or its derivatives, benzoquinone or its derivatives, naphthoquinone or its derivatives, anthraquinone or its derivatives, tetracyanoanthraquinodimethane or its derivatives, fluorenone derivatives, diphenyldicyanoethylene or its derivatives, diphenoquinone derivatives, metal complexes of 8-hydroxyquinoline or its derivatives, polyquinoline or its derivatives, polyquinoxaline or its derivatives, polyfluorene or its derivatives, and the like.

Specifically exemplified are those described in JP-A Nos. 63-70257, 63-175860, 2-135359, 2-135361, 2-209988, 3-37992 and 3-152184, and the like.

Of them, preferable are oxadiazole derivatives, benzoquinone or its derivatives, anthraquinone or its derivatives, or metal complexes of 8-hydroxyquinoline or its derivatives, polyquinoline or its derivatives, polyquinoxaline or its derivative and polyfluorene or its derivatives, and further preferable are 2-(4-biphenyl)-5-(4-t-butylphenyl)-1,3,4-oxadiazole, benzoquinone, anthraquinone, tris(8-quinolinol)aluminum and polyquinoline.

The method for forming an electron transport layer is not particularly restricted, and in the case of an electron transport material of low molecular weight, exemplified is a method of vacuum vapor deposition from a powder or a method of film formation from a solution or molten condition, and in the case of an electron transport material of high molecular weight, exemplified is a method of film formation from a solution or molten condition, respectively. In film formation from a solution or molten condition, a polymer binder may be used together.

The solvent to be used for film formation from a solution is not particularly restricted providing it dissolves an electron transport material and/or a polymer binder. Exemplified as the solvent are chlorine-based solvents such as chloroform, methylene chloride, dichloroethane and the like, ether-based solvents such as tetrahydrofuran and the like, aromatic hydrocarbon-based solvents such as toluene, xylene and the like, ketone-based solvents such as acetone, methyl ethyl ketone and the like, and ester-based solvents such as ethyl acetate, butyl acetate, ethyl cellosolve acetate and the like.

As the method of film formation from a solution or molten condition, application methods such as a spin coat method, casting method, micro gravure coat method, gravure coat method, bar coat method, roll coat method, wire bar coat method, dip coat method, spray coat method, screen printing method, flexographic printing method, offset printing method, inkjet printing method and the like can be used.

As the polymer binder to be mixed, those not extremely disturb charge transport are preferable, and those manifesting no strong absorption for visible light are suitably used. As the polymer binder, poly(N-vinylcarbazole), polyaniline or its derivatives, polythiophene or its derivatives, poly(p-phenylenevinylene) or its derivatives, poly(2,5-thienylenevinylene) or its derivatives, polycarbonate, polyacrylate, polymethyl acrylate, polymethyl methacrylate, polystyrene, polyvinyl chloride, polysiloxane and the like are exemplified.

Regarding the thickness of an electron transport layer, the optimum value varies depending on the material to be used and may be advantageously selected so that the driving voltage and emitting efficiency manifest suitable values, and at least thicknesses causing no generation of pin holes are necessary, and when the thickness is too large, the driving voltage of a device increases undesirably. Therefore, the thickness of the electron transport layer is, for example, from 1 nm to 1 μm, preferably 2 nm to 500 nm, further preferably 5 nm to 200 nm.

As the base plate for forming a polymer LED of the present invention, those not deforming in forming an electrode and forming a layer of an organic substance may be permissible, and exemplified are glass, plastics, polymer films, silicon base plates and the like. In the case of an opaque base plate, it is preferable that an opposite electrode is transparent or semi-transparent.

In the polymer LED of the present invention, it is usual that at least one of electrodes composed of an anode and a cathode is transparent or semi-transparent, and it is preferable that the anode side is transparent or semi-transparent. As the material of the anode, electrically conductive metal oxide membranes, semi-transparent metal thin membranes and the like are used. Specifically used are membranes (NESA and the like) manufactured using indium oxide, zinc oxide, tin oxide, and complexes thereof: indium-tin-oxide (1TO), indium-zinc-oxide and the like, or gold, platinum, silver, copper and the like, and preferable are ITO, indium-zinc-oxide and tin oxide. As the manufacturing method, a vacuum vapor deposition method, sputtering method, ion plating method, plating method and the like are mentioned. As the anode, organic transparent conductive membranes of polyaniline or derivatives thereof, polythiophene or derivatives thereof, and the like may be used.

The thickness of an anode can be appropriately selected taking light transmittance and electric conductivity into consideration, and it is, for example, 10 nm to 10 μm, preferably 20 nm to 1 μm, further preferably 50 nm to 500 nm.

For facilitating charge injection, a layer made of a phthalocyanine derivative, conductive polymer, carbon and the like, or a layer made of a metal oxide, metal fluoride, organic insulation material and the like and having an average thickness of 2 nm or less may be provided on an anode.

As the material of a cathode to be used in a polymer LED of the present invention, materials of small work function are preferable. For example, metals such as lithium, sodium, potassium, rubidium, cesium, beryllium, magnesium, calcium, strontium, barium, aluminum, scandium, vanadium, zinc, yttrium, indium, cerium, samarium, europium, terbium, ytterbium and the like, alloys composed of two or more of them, or alloys composed of one or more of them and one or more of gold, silver, platinum, copper, manganese, titanium, cobalt, nickel, tungsten and tin, and, graphite or graphite interlaminar compound and the like are used. Examples of the alloys include magnesium-silver alloy, magnesium-indium alloy, magnesium-aluminum alloy, indium-silver alloy, lithium-aluminum alloy, lithium-magnesium alloy, lithium-indium alloy, calcium-aluminum alloy and the like. A cathode may have a lamination structure composed of two or more layers.

The thickness of a cathode can be appropriately selected taking electric conductivity and durability into consideration, and it is, for example, 10 nm to 10 μm, preferably 20 nm to 1 μm, further preferably 50 nm to 500 nm.

As the method for manufacturing a cathode, a vacuum vapor deposition method, sputtering method, lamination method of thermo-compressing a metal thin membrane, and the like are used. Further, a layer composed of a conductive polymer or a layer composed of a metal oxide, metal fluoride, organic insulation material and the like and having an average thickness of 2 nm or less may be provided between a cathode and an organic substance layer, and after manufacturing a cathode, a protective layer for protecting the polymer LED may be mounted. For using the polymer LED stably for a long period of time, it is preferable to mount a protective layer and/or protective cover, for protecting a device from outside.

As the protective layer, polymer compounds, metal oxides, metal fluorides, metal borides and the like can be used. As the protective cover, glass plates, plastic plates on which a water permeability lowering treatment has been performed, and the like can be used, and a method is suitably used in which the cover is sealed by pasting with a device base plate with a thermosetting resin or photo-curing resin. When a space is maintained using a spacer, injuring of a device can be easily prevented. If an inert gas such as nitrogen or argon is filled in the space, oxidation of a cathode can be prevented, and further by placing a desiccant such as barium oxide and the like in the space, a damage on a device by moisture adsorbed in a production process can be easily suppressed. Of them, one or more strategies are preferably adopted.

The polymer LED of the present invention can be used as a sheet light source, segment display, dot matrix display, back light of a liquid crystal display, and the like.

For obtaining light emission in the form of sheet using a polymer LED of the present invention, it may be advantageous that a sheet anode and a sheet cathode are arranged so as to overlap. For obtaining pattern light emission, there are a method in which a mask having a pattern window is placed on the surface of the above-described sheet light emitting device, a method an organic substance layer at no-emission parts is formed with extremely large thickness to give substantially no-emission, and a method in which either a cathode or an anode or both electrodes are formed into a pattern. When a pattern is formed by any of these methods and some electrodes are arranged so that independent On/OFF is possible, then, a display of segment type which can display numbers, letters, simple marks and the like is obtained. Further, for obtaining a dot matrix device, it may be advantageous that both an anode and a cathode are formed in a stripe and arranged so as to cross. Partial color displaying and multi-color displaying are made possible by a method in which several kinds of light emitting polymers showing different emission colors are painted separately, or a method in which a color filter or a light emission-converting filter is used. In the case of the dot matrix device, passive driving is also possible, and active driving in combination with TFT and the like may also be permissible. These displays can be used as a display of computers, televisions, mobile terminals, mobile telephones, car navigations, video camera view finders and the like.

Further, the above-described sheet light emission device is of self luminous thin type, and can be suitably used as a sheet light source for back light of a liquid crystal display, or a sheet illumination light source. Furthermore, when a flexible base plate is used, it can be used also as a curved light source or display.

Examples for illustrating the present invention further in detail will be shown below, but the present invention is not limited to them.

The number-average molecular weight reduced by polystyrene was measured by SEC.

Column: TOSOH TSKgel SuperHM-H (two)+TSKgel SuperH2000 (4.6 mm I.d.×15 cm),

Detector: RI (SHIMADZU RID-10A) was used.

Tetrahydrofuran (THF) was used as a mobile phase.

### SYNTHESIS EXAMPLE 1

### Synthesis of 4-t-butyl-2,6-dimethylbromobenzene

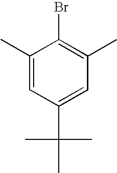

Under an inert atmosphere, into a 500 ml three-necked flask was placed 225 g of acetic acid, and 24.3 g of 5-t-butyl-m-xylene was added. Subsequently, 31.2 g of bromine was added, then, reacted at 15 to 20° C. for 3 hours.

The reaction liquid was added to 500 ml of water and the deposited precipitate was filtrated. The precipitate was washed with 250 ml of water twice, to obtain 34.2 g of white solid.

¹H-NMR (300 MHz/CDCl₃):

δ(ppm) 1.3 [s, 9H], 2.4 [s, 6H], 7.1 [s, 2H]

MS (FD⁺) M⁺ 241

### SYNTHESIS EXAMPLE 2

### Synthesis of N,N′-diphenyl-N,N′-bis(4-t-butyl-2,6-dimethylphenyl)-1,4-phenylenediamine

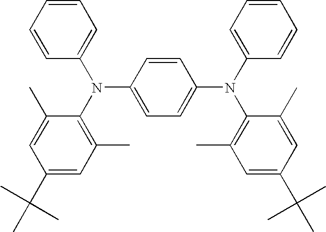

Under an inert atmosphere, into a 100 ml three-necked flask was placed 36 ml of deaerated dehydrated toluene, and 0.63 g of tri(t-butyl)phosphine was added. Subsequently, 0.41 g of tris(dibenzylideneacetone)dipalladium, 9.6 g of the above-described 4-t-butyl-2,6-dimethylbromobenzene, 5.2 g of t-butoxysodium and 4.7 g of N,N′-diphenyl-1,4-phenylenediamine were added, then, reacted at 100° C. for 3 hours.

The reaction liquid was added to 300 ml of saturated saline and extracted with 300 ml of chloroform warmed at about 5° C. The solvent was distilled off, then, 100 ml of toluene was added and the mixture was heated until dissolution of solid and allowed to cool, then, the precipitate was filtrated to obtain 9.9 g of white solid.

### SYNTHESIS EXAMPLE 3

### Synthesis of N,N′-bis(4-bromophenyl)-N,N′-bis(4-t-butyl-2,6-dimethylphenyl)-1,4-phenylenediamine

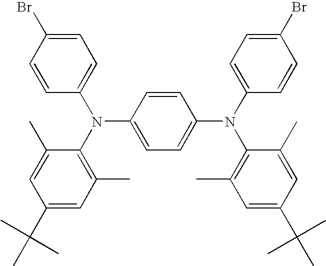

Under an inert atmosphere, into a 1000 ml three-necked flask was placed 350 ml of dehydrated N,N-dimethylformamide, and 5.2 g of the above-described N,N′-diphenyl-N,N′-bis(4-t-butyl-2,6-dimethylphenyl)-1,4-phenylenediamine was dissolved, then, N-bromosuccinimide 3.5 g/N,N-dimethylformamide solution was dropped in an ace bath, and reacted over night and day.

150 ml of water was added to the reaction liquid, and the deposited precipitate was filtrated and washed with 50 ml of methanol twice to obtain 4.4 g of white solid.

¹H-NMR (300 MHz/THF-d8):

δ(ppm)=1.3 [s, 18H], 2.0 [s, 12H], 6.6˜6.7 [d, 4H], 6.8˜6.9 [br, 4H], 7.1 [s, 4H], 7.2˜7.3 [d, 4H]

MS (FD⁺) M⁺ 738

### SYNTHESIS EXAMPLE 4

### Synthesis of Compound A

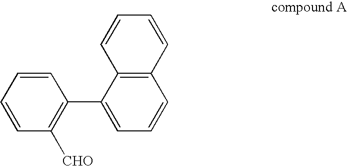

Under an inert atmosphere, into a 300 ml three-necked flask was placed 5.00 g (29 mmol) of 1-naphthaleneboronic acid, 6.46 g (35 mmol) of 2-bromobenzaldehyde, 10.0 g (73 mmol) of potassium carbonate, 36 mol of toluene and 36 ml of ion exchanged water, and argon was bubbled through the mixture for 20 minutes while stirring at room temperature. Subsequently, 16.8 mg (0.15 mmol) of tetrakis(triphenylphosphine)palladium was added, and further, argon was bubbled through the mixture for 10 minutes while stirring at room temperature. The mixture was heated up to 100° C. and reacted for 25 hours. The reaction mixture was cooled down to room temperature, then, an organic layer was extracted with toluene, and dried over sodium sulfate, then, the solvent was distilled off. Purification by a silica gel column using a toluene:cyclohexane=1:2 mixed solvent as a developing solvent was performed to obtain 5.18 g (yield: 86%) of compound A as white crystal.

¹H-NMR (300 MHz/CDCl₃):

δ7.39˜7.62 (m, 5H), 7.70 (m, 2H), 7.94 (d, 2H), 8.12 (dd, 2H), 9.63 (s, 1H)

MS (APCI (+)): (M+H)⁺ 233

### SYNTHESIS EXAMPLE 5

### Synthesis of Compound B

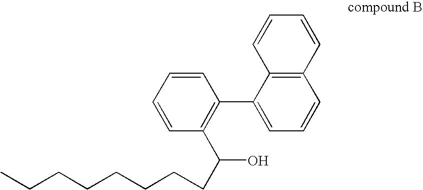

Under an inert atmosphere, into a 300 ml three-necked flask was placed 8.00 g (34.4 mmol) of compound A and 46 ml of dehydrated THF, and the mixture was cooled down to −78° C. Subsequently, 52 ml of n-octylmagnesium bromide (1.0 mol/l THF solution) was dropped over 30 minutes. After completion of dropping, the solution was heated up to 0° C., and stirred for 1 hour, then, heated to room temperature and stirred for 45 minutes. In an ice bath, 20 ml of 1 N hydrochloric acid was added to the solution to terminate the reaction, and an organic layer was extracted with ethyl acetate, and dried over sodium sulfate. The solvent was distilled off, then, purification by a silica gel column using a toluene:hexane=10:1 mixed solvent as a developing solvent was performed to obtain 7.64 g (yield: 64%) of compound B as pale yellow oil. Though two peaks were observed in HPLC measurement, the product was judged to be a mixture of isomers since the mass numbers thereof were identical in LC-MS measurement.

### SYNTHESIS EXAMPLE 6

### Synthesis of Compound C

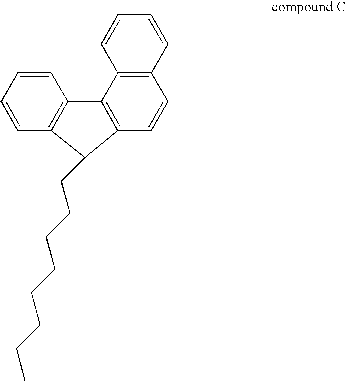

Under an inert atmosphere, into a 500 ml three-necked flask was placed 5.00 g (14.4 mmol) of compound B and 74 ml of dehydrated dichloromethane, and the mixture was stirred at room temperature for dissolution. Subsequently, an etherate complex of boron trifluoride was dropped at room temperature over 1 hour, and after completion of dropping, the mixture was stirred at room temperature for 4 hours. 125 ml of ethanol was added slowly while stirring, and when heat generation stopped, an organic layer was extracted with chloroform, washed with water twice and dried over magnesium sulfate. The solvent was distilled off, then, purification by a silica gel column using hexane as a developing solvent was performed to obtain 3.22 g (yield: 68%) of compound C as colorless oil.

¹H-NMR (300 MHz/CDCl₃):

δ0.90 (t, 3H), 1.03˜1.26 (m, 14H), 2.13 (m, 2H), 4.05 (t, 1H)>7.35 (dd, 1H), 7.46˜7.50 (m, 2H), 7.59˜7.65 (m, 3H), 7.82 (d, 1H), 7.94 (d, 1H), 8.35 (d, 1H), 8.75 (d, 1H)

MS (APCI (+)): (M+H)⁺ 329

### SYNTHESIS EXAMPLE 7

### Synthesis of Compound D

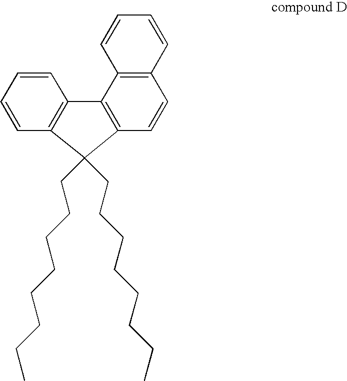

Under an inert atmosphere, into a 200 ml three-necked flask was placed 20 ml of ion exchanged water, and 18.9 g (0.47 mol) of sodium hydroxide was added portion-wise while stirring to cause dissolution thereof. The aqueous solution was cooled to room temperature, then, 20 ml of toluene, 5.17 g (15.7 mmol) of compound C and 1.52 g (4.72 mmol) of tributylammonium bromide were added, and the temperature was raised to 50° C. n-octyl bromide was dropped, and after completion of dropping, the mixture was reacted at 50° C. for 9 hours. After completion of the reaction, an organic layer was extracted with toluene, washed with water twice and dried over sodium sulfate. Purification by a silica gel column using hexane as a developing solvent was performed to obtain 5.12 g (yield: 74%) of compound D as yellow oil.

¹H-NMR (300 MHz/CDCl₃):

δ0.52 (m, 2H), 0.79 (t, 6H), 1.00˜1.20 (m, 22H), 2.05 (t, 4H), 7.34 (d, 1H), 7.40˜7.53 (m, 2H), 7.63 (m, 3H), 7.83 (d, 1H), 7.94 (d, 1H), 8.31 (d, 1H)<8.75 (d, 1H)

MS (APCI (+)): (M+H)⁺ 441

### SYNTHESIS EXAMPLE 8

### Synthesis of Compound E

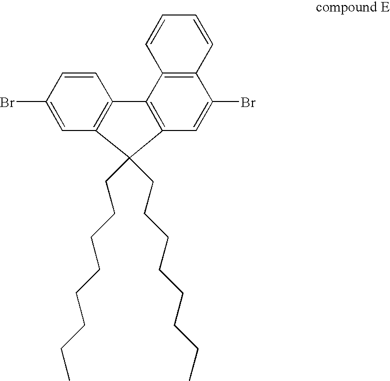

Under an air atmosphere, into a 50 ml three-necked flask was placed 4.00 g (9.08 mmol) of compound D and 57 ml of an acetic acid:dichloromethane=1:1 mixed solvent, and the mixture was stirred at room temperature for dissolution. Subsequently, 7.79 g (20.0 mmol) of benzyltrimethylammonium tribromide was added and zinc chloride was added until complete dissolution of benzyltrimethylammonium tribromide while stirring. The mixture was stirred at room temperature for 20 hours, then, 10 ml of a 5% sodium hydrogen sulfite aqueous solution was added to terminate the reaction, and an organic layer was extracted with chloroform, washed with an aqueous potassium carbonate solution twice, and dried over sodium sulfate. Purification by a flush column using hexane as a developing solvent was performed twice, then, re-crystallization was performed using an ethanol-hexane=1:1, then, 10:1 mixed solvent, to obtain 4.13 g (yield: 76%) of compound E as white crystal.

¹H-NMR (300 MHz/CDCl₃):

δ0.60 (m, 2H), 0.91 (t, 6H), 1.01˜1.38 (m, 22H), 2.09 (t, 4H), 7.62˜7.75 (m, 3H), 7.89 (s, 1H), 8.20 (d, 1H), 8.47 (d, 1H), 8.72 (d, 1H)

MS (APPI (+)): (M+H)⁺ 598

### SYNTHESIS EXAMPLE OF COMPOUND F

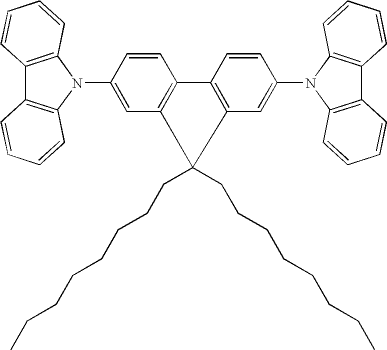

Into a 100 mL three-necked flask was weighed Pd(OAc)₂ (123 mg, 0.54 mmol), P(t-Bu)₃.HBF₄ (476 mg, 16.4 mmol) and Cs₂CO₃ (2.67 g, 82.1 mmol) and an argon atmosphere was made.

Dehydrated xylene (50 mL) was introduced by a syringe, and the mixture was stirred at room temperature for 2 hours. Carbazole (2.74 g, 16.4 mmol) and 2,7-dibromo-9,9-di-n-octylfluorene (3 g, 5.47 mmol) were added into the flask and the mixture was heated at 120° C. for 8 hours.

After completion of the reaction, the reaction liquid was cooled down to room temperature, then, the solid was filtrated. The filtrate was concentrated and dried to solid, and purified by silica gel chromatography (developing solvent: CHCl₃/hexane (1:3, v/v)) to obtain compound F (2.9 g, 74.4%) as white solid in the form of film.

¹H-NMR (300 MHz/CDCl₃):

δ0.79˜0.89 (m, 10H), 1.15˜1.27 (m, 20H), 2.01˜2.07 (m, 4H), 7.30˜7.35 (m, 4H), 7.41˜7.49 (m, 8H), 7.59˜7.62 (m, 4H), 7.98 (d J=7.5 Hz, 1H), 8.19 (d J=8.4 Hz, 4H)

MS (APCI (+)): (M+H)⁺ 721

### SYNTHESIS EXAMPLE 9

### Synthesis of Polymer Compound 1

Compound E (8.0 g) and 2,2′-bipyridyl (5.9 g) were dissolved in 300 mL of dehydrated tetrahydrofuran, then, nitrogen was bubbled through the solution to purge an atmosphere in the system with nitrogen. Under a nitrogen atmosphere, this solution was heated up to 60° C., and bis(1,5-cyclooctadiene)nickel(0){Ni(COD)₂} (10.4 g, 0.038 mol) was added, and reacted for 5 hours. After the reaction, this solution was cooled to room temperature (about 25° C.), and dropped into a mixed solution of 25% ammonia water 40 mL/methanol 300 mL/ion exchanged water 300 mL and stirred for 30 minutes, then, the deposited precipitate was filtrated and air-dried. Then, the product was dissolved in 400 mL of toluene before conducting filtration, and the filtrate was purified by passing through an alumina column, and about 300 mL of 1 N hydrochloric acid was added and the mixture was stirred for 3 hours, an aqueous layer was removed, about 300 mL of 4% ammonia water was added to an organic layer, and the mixture was stirred for 2 hours, then, an aqueous layer was removed. About 300 mL of ion exchanged water was added to an organic layer, and the mixture was stirred for 1 hour, then, an aqueous layer was removed. About 100 mL of methanol was dropped into an organic layer and the mixture was stirred for 1 hour, subsequently, the solution was allowed to stand still, then, the supernatant was removed by decantation. The resultant precipitate was dissolved in 100 mL of toluene, dropped into about 200 mL of methanol and the mixture was stirred for 1 hour, and the resultant solution was filtrated and dried under reduced pressure for 2 hours. The yield of the resultant copolymer was 4.1 g (hereinafter, referred to as polymer compound 1). The polystyrene-reduced average molecular weight and the polystyrene-reduced weight-average molecular weight of polymer compound 1 were Mn=1.5×10⁵ and Mw=2.7×10⁵, respectively (mobile phase: tetrahydrofuran).

### SYNTHESIS EXAMPLE 10

### Synthesis of Polymer Compound 2

Compound E (0.65 g), N,N′-bis(4-bromophenyl)-N,N′-bis(4-t-butyl-2,6-dimethylphenyl)-1,4-phenylenediamine (0.34 g) and 2,2′-bipyridyl (0.58 g) were dissolved in 100 mL of dehydrated tetrahydrofuran, then, nitrogen was bubbled through the solution to purge an atmosphere in the system with nitrogen. Under a nitrogen atmosphere, to this solution was added bis(1,5-cyclooctadiene)nickel(0){Ni(COD)₂} (1.0 g) and the mixture was heated up to 60° C. and reacted for 3 hours while stirring. After the reaction, this solution was cooled to room temperature (about 25° C.), and dropped into a mixed solution of 25% ammonia water 10 mL/methanol about 100 mL/ion exchanged water about 100 mL and stirred for 1 hour, then, the deposited precipitate was filtrated and dried under reduced pressure for 3 hours, then, dissolved in 50 mL of toluene before conducting filtration, and the filtrate was purified by passing through an alumina column, and about 50 mL of 4% ammonia water was added and the mixture was stirred for 2 hours, then, an aqueous layer was removed. About 50 mL of ion exchanged water was added to an organic layer, and the mixture was stirred for 1 hour, then, an aqueous layer was removed. An organic layer was dropped into about 100 mL of methanol and the mixture was stirred for 1 hour, subsequently, the solution was allowed to stand still, then, the supernatant was removed by decantation. The resultant precipitate was dissolved in 50 mL of toluene, dropped into about 200 mL of methanol and the mixture was stirred for 1 hour, and the resultant solution was filtrated and dried under reduced pressure for 2 hours. The yield of the resultant copolymer was 390 mg (hereinafter, referred to as polymer compound 2). The polystyrene-reduced average molecular weight and the polystyrene-reduced weight-average molecular weight of polymer compound 2 were Mn=1.6×10⁴ and Mw=7.4×10⁴, respectively (mobile phase: tetrahydrofuran).

### SYNTHESIS EXAMPLE 11

### Synthesis of Compound G

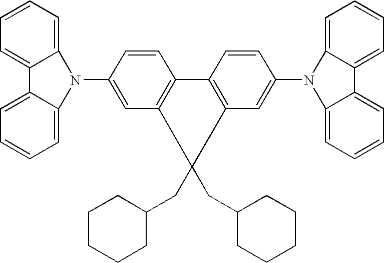

Into a 50 mL three-necked flask was charged Pd(OAc)₂ (0.007 g, 0.03 mmol), carbazole (0.75 g, 4.5 mmol), 2,7-dibromo-9,9-di-cyclohexylmethylfluorene (0.77 g, 1.5 mmol) and K₂CO₃ (1.24 g, 9 mmol), purging with argon under reduced pressure was performed three times. Then, dehydrated toluene (16 mL) was added by a syringe, and again, purging with argon under reduced pressure was performed. This mass was heated up to 70 to 75° C., and (t-Bu)P (0.015 g, 0.075 mmol) was added and the mixture was heated and stirred for 9 hours while heating at 105 to 107° C. After completion of the reaction, the reaction liquid was cooled down to room temperature and solid was filtrated. The residue on filter paper was washed with chloroform (50 mL) and the filtrate was concentrated and dried to solid. Purification by silica gel chromatography {developing solvent: chloroform:hexane (1:10, v/v), 0.1% triethylamine added} was performed to obtain compound G (0.6 g, yield: 59.2%) as white solid.

¹H-NMR (270 MHz/CDCl₃):

δ0.747 (m, 6H), 0.934 (m, 6H), 1.165 (m, 4H), 1.459 (m, 6H), 1.983 (d, 4H), 7.382 (m, 12H), 7.583 (m, 4H), 8.006 (m, 2H), 8.187 (d, 4H)

### SYNTHESIS EXAMPLE 12

### Synthesis of Compound H

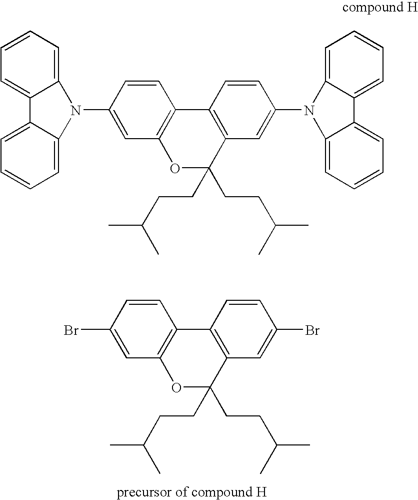

Into a 50 mL three-necked flask was charged Pd(OAc)₂ (0.007 g, 0.03 mmol), carbazole (0.80 g, 4.8 mmol), the above-described precursor (0.77 g, 1.6 mmol) synthesized according to descriptions of Japanese Patent Application No. 2003-343244 and (1.33 g, 9.6 mmol), purging with argon under reduced pressure was performed three times. Then, dehydrated toluene (16 mL) was added by a syringe, and again, purging with argon under reduced pressure was performed. This mass was heated up to 70 to 75° C., and (t-Bu)P (0.016 g, 0.08 mmol) was added and the mixture was heated and stirred for 6 hours while heating at 105 to 107° C. After completion of the reaction, the reaction liquid was cooled down to room temperature and solid was filtrated. The residue on filter paper was washed with chloroform (50 mL) and the filtrate was concentrated and dried to solid. Purification by silica gel chromatography {developing solvent: chloroform:hexane (1:10, v/v), 0.1% triethylamine added} was performed to obtain 1.05 g of white solid. To this white solid was added 30 g of methanol and the mixture was stirred for 30 minutes under reflux and cooled down to room temperature, then, filtrated and the cake was washed with 20 mL of methanol. The cake was dried at 80° C. under reduced pressure to obtain compound H (0.87 g, yield: 82.8%).

¹H-NMR (270 MHz/CDCl₃):

δ0.882 (m, 12H), 1.407 (m, 6H), 1.992 (m, 4H), 7.597 (m, 16H), 8.069 (m, 2H), 8.174 (m, 4H)

### SYNTHESIS EXAMPLE 13

### Synthesis of Compound I

### (1) Synthesis of Precursor

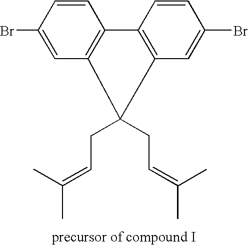

Into a 500 mL three-necked flask was charged NaOH (48.9 g, 1.2 mol) and water (89.1 g) to dissolve NaOH. The temperature was controlled at 50° C., then, 2,7-dibromofluorene (12.96 g, 40 mmol), toluene (74.8 g) and tetra-n-butylammonium bromide (7.74 g, 24 mmol) were charged.

While stirring, 1-chloro-3-methyl-2-butene (12.55 g, 120 mmol) was dropped at 52 to 55° C. over 40 minutes. Thereafter, the mixture was stirred for 3 hours at 50 to 55° C. After completion of the reaction, the solution was cooled down to room temperature and toluene (50 mL) was added, and an aqueous layer was separated. An oil layer was washed with water (100 mL) for four times, then, concentrated to obtain 19.0 g of solid. This solid was dissolved in ethanol (57 g) at 80° C., the deposited crystal was filtrated at temperatures of 5° C. or lower and washed with ethanol (30 mL), then, dried to obtain 15.3 g of a dry cake. Re-crystallization was further performed twice at cake/hexane/ethanol=1/0.5/1.5 (weight ratio), then, the crystal was dried to obtain a white precursor (10.3 g, yield: 55.9%).

¹H-NMR (300 MHz/CDCl₃):

δ 1.42 (s, 6H), 1.48 (s, 6H), 2.525 (d, 4H), 4.625 (t, 2H), 7.465 (m, 6H)

### (2) Synthesis of Compound I

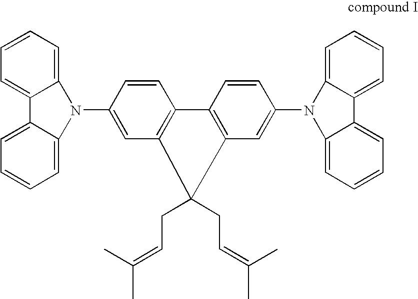

Pd(OAc)₂ (0.045 g, 0.2 mmol), carbazole (5.02 g, 30 mmol), precursor (4.60 g, 10 mmol) and K₂CO₃ (8.29 g, 60 mmol) were charged and purging with argon under reduced pressure was performed three times. Then, dehydrated toluene (50 mL) was added by a syringe, and again, purging with argon under reduced pressure was performed.

This mass was heated up to 70 to 75° C., and (t-Bu)P (0.10 g, 0.5 mmol) was added and the mixture was heated and stirred for 11 hours while heating at 105 to 107° C. After completion of the reaction, the reaction liquid was cooled down to room temperature and solid was filtrated. The residue on filter paper was washed with chloroform (100 mL) and the filtrate was concentrated and dried to solid to obtain a solid component (8.6 g).

Re-crystallization was performed under a condition of solid component/chloroform/ethanol=1/5/5 (weight ratio) to obtain a dry cake (6.04 g).

Purification by silica gel chromatography {developing solvent: chloroform:hexane (1:10, v/v), 0.1% triethylamine added} was performed to obtain white solid (6.00 g). Further, re-crystallization was performed twice under a condition of white solid/chloroform/hexane=1/3/10 (weight ratio) and the crystal was dried under reduced pressure at 80° C., to obtain compound I (5.10 g, yield: 80.5%).

¹H-NMR (270 MHz/CDCl₃):

δ 1.513 (s, 6H), 1.551 (s, 6H), 2.730 (m, 4H), 4.818 (m, 2H), 7.314 (m, 4H), 7.446 (m, 8H), 7.586 (m, 4H), 7.976 (d, 2H), 8.185 (d, 4H)

### SYNTHESIS EXAMPLE 14

### Synthesis of Compound J

### (1) Synthesis of Precursor

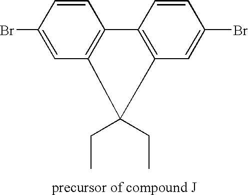

Into a 500 mL three-necked flask was charged NaOH (48.0 g, 1.2 mol) and water (89.1 g) to dissolve NaOH. The temperature was controlled at 50° C., then, 2,7-dibromofluorene (12.96 g, 40 mmol), toluene (64.8 g) and tetra-n-butylammonium bromide (7.74 g, 24 mmol) were charged.

While stirring, ethyl bromide (6.54 g, 60 mmol) was dropped at 52 to 55° C. over 30 minutes. Thereafter, the mixture was stirred for 7 hours at 50 to 55° C. After completion of the reaction, the solution was cooled down to room temperature and toluene (50 mL) was added, and an aqueous layer was separated. An oil layer was washed with water (100 mL) for four times, then, concentrated to obtain 15.2 g of solid. This solid was washed with ethanol (45 g) at 75 to 80° C. for 1 hour, cooled down to room temperature, then, filtrated, and washed with ethanol (30 mL). The washed product was dried to obtain a dry cake (15.2 g). Re-crystallization was further performed three times at cake/chloroform/hexane=1/2/4 (weight ratio), then, dried to obtain a white precursor (4.90 g, yield: 32.2%).

¹H-NMR (270 MHz/CDCl₃):

δ0.317 (t, 6H), 1.991 (q, 4H), 7.493 (m, 6H)

### (2) Synthesis of Compound J

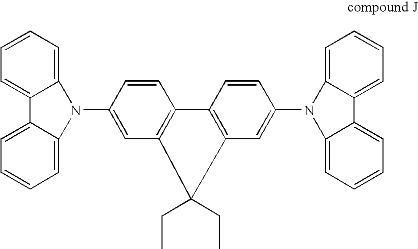

Pd(OAc)₂ (0.045 g, 0.2 mmol), carbazole (5.02 g, 30 mmol), precursor (3.80 g, 10 mmol) and K₂CO₃ (8.29 g, 60 mmol) were charged and purging with argon under reduced pressure was performed three times. Then, 40 mL of dehydrated toluene was added by a syringe, and again, purging with argon under reduced pressure was performed. This mass was heated up to 70 to 75° C., and (t-Bu)P (0.10 g, 0.5 mmol) was added and the mixture was heated and stirred for 10.5 hours while heating at 105 to 107° C. After completion of the reaction, the reaction liquid was cooled down to room temperature and solid was filtrated. The residue on filter paper was washed with chloroform (150 mL) and the filtrate was concentrated and dried to solid to obtain a solid component (6.85 g).

Re-crystallization was performed under a condition of solid component/chloroform/ethanol=1/5/5 (weight ratio) to obtain a dry cake (5.45 g). This cake was dissolved in chloroform (24 g) and hexane (36 g) was added, and re-crystallization was performed to obtain a dry cake (5.00 g). Purification by silica gel chromatography {developing solvent: chloroform:hexane (1:5, v/v), 0.1% triethylamine added} was performed to obtain white solid (5.00 g). Further, the while solid was dissolved in chloroform (24 g) and hexane (36 g) was added, an re-crystallization was performed to obtain compound J (4.55 g, yield: 82.4%).

¹H-NMR (270 MHz/CDCl₃):

δ 0.559 (t, 6H), 2.101 (m, 4H), 7.334 (m, 4H), 7.456 (m, 8H), 7.594 (m, 4H), 8.087 (d, 2H), 8.185 (d, 4H)

### SYNTHESIS EXAMPLE 15

### Synthesis of Compound K

### Synthesis of Oxetane Unit

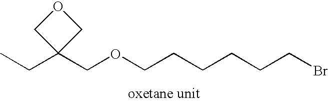

Into a 1 L three-necked flask purged with argon was placed 163 ml of ion exchanged water, and 85.2 g (2.13 mol) of sodium hydroxide was added portion-wise and stirred for dissolution. Subsequently, 12.5 g (0.04 mol) of tetrabutylammonium bromide was placed, and 15 g (0.13 mol) of 3-ethyl-3-oxetanemethanol, 94.5 g (0.39 mol) of 1,6-dibromohexane and 128 ml of hexane were added and the mixture was reacted at room temperature for 9 hours, then, heated up to 80° C. and reacted for 1 hour. After cooling down to room temperature, an organic layer was extracted with hexane and dried over sodium sulfate, then, the solvent was distilled off. Purification by distillation under reduce pressure was performed to obtain 32.4 g of an oxetane unit in the form of colorless transparent oil.

### (1) Synthesis of Precursor

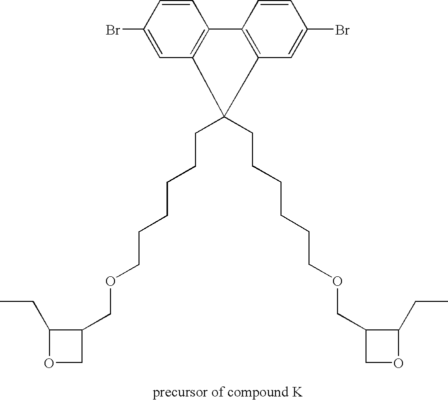

Into a 200 mL three-necked flask was charged NaOH (12.0 g, 0.3 mol) and water (22.3 g) to dissolve NaOH. The temperature was controlled at 50° C., then, 2,7-dibromofluorene (3.24 g, 10 mmol), toluene (13.9 g) and tetra-n-butylammonium bromide (0.97 g, 3 mmol) were charged.

While stirring, a solution prepared by dissolving the above-described oxetane unit (6.98 g, 25 mmol) in toluene (7.0 g) was dropped at 52 to 55° C. over 25 minutes. Thereafter, the mixture was stirred for 8 hours at 50 to 55° C. After completion of the reaction, the solution was cooled down to room temperature and toluene (100 mL) was added, and an aqueous layer was separated. An oil layer was washed with water (50 mL) for four times, then, concentrated to obtain 9.0 g of viscous oil (partially crystallized). To this mass was added methanol (20 g) and the crystal was filtrated off. The filtrate was concentrated to obtain viscous oil (9.0 g). Purification by silica gel chromatography {developing solvent: chloroform:hexane (1:1, v/v), 0.1% triethylamine added} was performed three times to obtain a precursor (1.82 g, yield: 25.2%).

¹H-NMR (270 MHz/CDCl₃):

δ 0.594 (m, 4H), 0.850 (t, 6H), 1.096 (m, 8H), 1.380 (m, 4H), 1.693 (m, 4H), 1.914 (m, 4H), 3.332 (t, 4H), 3.458 (s, 4H), 4.326 (d, 4H), 4.408 (d, 4H), 7.486 (m, 6H)

### (2) Synthesis of Compound K

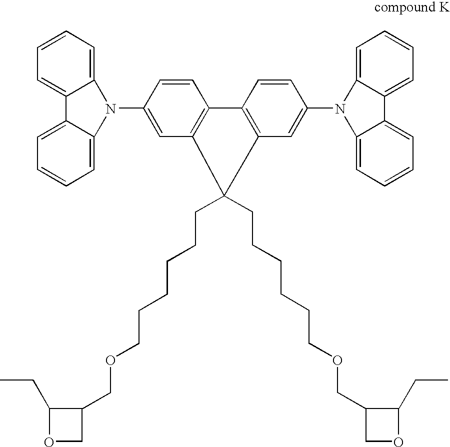

Pd(OAc)₂ (0.007 g, 0.03 mmol), carbazole (0.75 g, 4.5 mmol), precursor (1.08 g, 1.5 mmol) and K₂CO₃ (1.24 g, 9 mmol) were charged and purging with argon under reduced pressure was performed three times. Then, dehydrated toluene (16 mL) was added by a syringe, and again, purging with argon under reduced pressure was performed.

This mass was heated up to 70 to 75° C., and (t-Bu)P (0.015 g, 0.075 mmol) was added and the mixture was heated and stirred for 6 hours while heating at 105 to 107° C. After completion of the reaction, the reaction liquid was cooled down to room temperature and solid was filtrated. The residue on filter paper was washed with chloroform (50 mL) and the filtrate was concentrated and dried to solid to obtain a solid component (1.95 g). Purification by silica gel chromatography {developing solvent: chloroform:hexane (1:2, v/v), 0.1% triethylamine added} was performed twice to obtain compound K (0.92 g, yield: 68.6%) in the form of resinous solid.

¹H-NMR (270 MHz/CDCl₃):

δ0.809 (t, 4H), 0.901 (m, 6H), 1.194 (m, 8H), 1.446 (m, 4H), 1.658 (m, 4H), 2.089 (m, 4H), 3.338 (t, 4H), 3.442 (s, 4H), 4.299 (d, 4H), 4.367 (d, 4H), 7.366 (m, 4H), 7.456 (m, 8H), 7.608 (m, 4H), 7.992 (d, 2H), 8.187 (d, 4H)

### SYNTHESIS EXAMPLE 16

### Synthesis of 4-t-butyl-2,6-dimethylbromobenzene

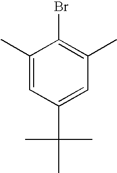

Under an inert atmosphere, into a 500 ml three-necked flask was placed 225 g of acetic acid, and 24.3 g of 5-t-butyl-m-xylene was added. Subsequently, 31.2 g of bromine was added, then, reacted at 15 to 20° C. for 3 hours.

The reaction liquid was added to 500 ml of water and the deposited precipitate was filtrated. The precipitate was washed with 250 ml of water twice, to obtain 34.2 g of white solid.

¹H-NMR (300 MHz/CDCl₃):

δ(ppm)=1.3 [s, 9H], 2.4 [s, 6H], 7.1 [s, 2H]

MS (FD⁺) M⁺241

### SYNTHESIS EXAMPLE 17

### Synthesis of N,N′-diphenyl-N,N′-bis(4-t-butyl-2,6-dimethylphenyl)-benzidine

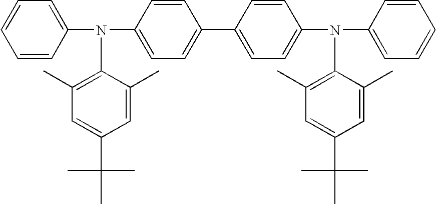

Under an inert atmosphere, into a 300 ml three-necked flask was placed 1660 ml of dehydrated toluene, and 275.0 g of N,N′-diphenylbenzidine and 449.0 g of 4-t-butyl-2,6-dimethylbromobenzene were added. Subsequently, 7.48 g of tris(dibenzylideneacetone)dipalladium and 196.4 g of t-butoxysodium were added, then, 5.0 g of tri(t-butyl)phosphine was added. Then, the mixture was reacted at 105° C. for 7 hours.

To the reaction liquid was added 2000 ml of toluene and the mixture was filtrated through cerite, and the filtrate was washed three times with 1000 ml of water, then, concentrated to 700 ml. To this was added 1600 ml of a toluene/methanol (1:1) solution, and the deposited crystal was filtrated and washed with methanol. 479.4 g of white solid was obtained.

### SYNTHESIS EXAMPLE 18

### Synthesis of N,N′-bis(4-bromophenyl)-N,N′-bis(4-t-butyl-2,6-dimethylphenyl)-benzidine

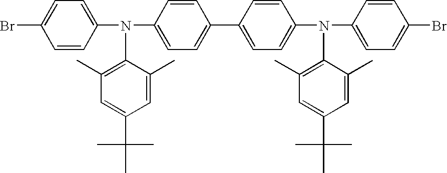

Under an inert atmosphere, into 4730 g of chloroform was dissolved 472.8 g of the above-described N,N′-diphenyl-N,N′-bis(4-t-butyl-2,6-dimethylphenyl)-benzidine, then, 281.8 g of N-bromosuccinimide was charged in 12-divided portions over 1 hour under shading in an ice bath, and reacted for 3 hours.

1439 ml of chloroform was added to the reaction liquid, and the mixture was filtrated, and the filtrate chloroform solution was washed with 2159 ml of 5% sodium thiosulfate, and toluene was distilled off to obtain a white crystal. The resultant white crystal was re-crystallized from toluene/ethanol, to obtain 678.7 g of a white crystal.

### SYNTHESIS EXAMPLE 19

### Synthesis of Polymer Compound 3

Compound E (5.25 g), N,N′-bis(4-bromophenyl)-N,N′-bis(4-t-butyl-2,6-dimethylphenyl)-benzidine (3.06 g) and 2,2′-bipyridyl (5.3 g) were dissolved in 226 mL of dehydrated tetrahydrofuran, then, nitrogen was bubbled through the solution to purge an atmosphere in the system with nitrogen. Under a nitrogen atmosphere, to this solution was added bis(1,5-cyclooctadiene)nickel(0){Ni(COD)₂} (9.30 g) and the mixture was heated up to 60° C. and reacted for 3 hours while stirring. After the reaction, this solution was cooled to room temperature (about 25° C.), and dropped into a mixed solution of 25% ammonia water 45 mL/methanol about 230 mL/ion exchanged water about 230 mL and stirred for 1 hour, then, the deposited precipitate was filtrated and dried under reduced pressure for 2 hours, then, dissolved in 400 mL of toluene before conducting filtration, and the filtrate was purified by passing through an alumina column, and about 400 mL of 5.2% hydrochloric acid water was added and the mixture was stirred for 3 hours, then, an aqueous layer was removed. Then, about 400 mL of 4% ammonia water was added and the mixture was stirred form 2 hours, then, an aqueous layer was removed. Further, to an organic layer was added about 400 mL of ion exchanged water and the mixture was stirred for 1 hour, then, an aqueous layer was removed. To an organic layer was added 80 mL of toluene, and the deposited precipitate was collected by decantation and dissolved in 200 ml of toluene, then, this was dropped into about 600 mL of methanol and the mixture was stirred for 1 hour, and the deposited precipitate was filtrated and dried under reduced pressure for 2 hours. The yield of the resultant copolymer (hereinafter, referred to as polymer compound 3) was 4.25 g. The polystyrene-reduced number-average molecular weight and the polystyrene-reduced weight-average molecular weight were Mn=2.5×10⁴ and Mw=8.0×10⁵, respectively (mobile phase: tetrahydrofuran).

### SYNTHESIS EXAMPLE 20

### Protection of Amino Group of 3,6-dibromocarbazole

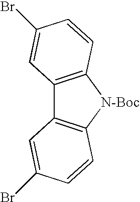

Under a nitrogen atmosphere, into a 1 L three-necked flask was added 93.4 g of 3,6-dibromocarbazole and 1.76 g of 4-dimethylaminopyridine, then, 467 ml of dehydrated tetrahydrofuran was added to dissolve them. 69.0 g of di-t-butyl dicarbonate was weighed into a dropping funnel and dropped over 1.5 hours at 20 to 23° C. under water cool. At the same temperature, the mixture was stirred for 1 hour. The reaction liquid was transferred to a 1 L eggplant-shaped flask and tetrahydrofuran was distilled off by an evaporator at 60° C. under reduced pressure. A crystal deposited during concentration. Next, the crystal was dried at 70° C. and 3 mmHg to obtain 122.4 g of a coarse cake. To the coarse cake was added 250 g of ethanol and the mixture was stirred for 1 hour under ref lux and cooled down to room temperature, then, filtrated. The wet cake was washed twice with 150 g of ethanol. To this wet cake was added 250 g of ethanol, and the same purification operation as for the coarse cake was conducted again, then, dried at 80° C. and 3 mmHg. 117.9 g of a slightly yellowish dry cake was obtained.

¹H-NMR (270 MHz/CDCl₃):

δ(ppm)=1.75 [s, 9H], 7.57 [d, 2H], 8.03 [s, 2H], 8.16 [d, 2H]

### SYNTHESIS EXAMPLE 21

### Synthesis of 3,6-di-n-butylcarbazole

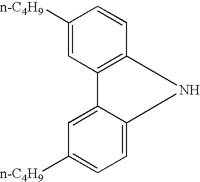

Under an argon atmosphere, into a 500 ml three-necked flask was added 25.5 g of the Boc compound synthesized in Synthesis Example 20, 41.5 g of potassium carbonate, 14.7 g of n-butylboronic acid, 96.7 g of ion exchanged water and 153 g of toluene. The pressure in the flask was reduced down to 40 mmHg at room temperature and the pressure was recovered with argon, and this operation was repeated three times, to purge an atmosphere in the flask with argon. The temperature was raised to 65° C. and 0.35 g of tetrakis(triphenylphosphine)palladium(0) was added. Next, 2.7 ml of a 10% toluene solution of tri-t-butylphosphine was weighed by a syringe and added. The temperature was raised and reacted under reflux for 3.5 hours at 84 to 85° C. After completion of the reaction, the solution was cooled down to 65° C., and the reaction liquid was transferred to a dropping funnel and an aqueous layer was separated. An oil layer was washed twice with 100 ml of ion exchanged water thermally-insulated at 60 to 65° C. An intermediate layer was produced, thus, washing with 100 ml of 10% saline was performed again at the same temperature. An oil layer was cooled down to room temperature, and anhydrous sodium sulfate was added in suitable amount and dehydrated at 20 to 23° C.

An oil layer was filtrated off, then, anhydrous sodium sulfate on filter paper was washed with 50 ml of toluene. The filtrate was concentrated by an evaporator and dried to solid finally at 70° C. and 3 mmHg. 23.4 g of an orange Boc compound crystal was obtained.

Under an argon atmosphere, into a 1 L three-necked flask was added 23.4 g of the Boc compound crystal and 494 ml of a 1 mol/L tetrahydrofuran solution of tetrabutylammonium fluoride. The mixture was heated and reacted for 66 hours under ref lux. The solution was cooled down to room temperature, then, the reaction mass was transferred to a 1 L eggplant-shaped flask and concentrated by an evaporator at 60° C. under reduced pressure to obtain 228.1 g of a concentrate. To the concentrate was added 500 ml of ion exchanged water, and the mixture was transferred to a 1 L dropping funnel and extracted twice with 200 ml of chloroform. The chloroform layer was transferred to a 1 L eggplant-shaped flask and concentrated by an evaporator at 60° C. under reduced pressure to obtain 61.5 g of a concentrate. Purification by silica gel chromatography {developing solvent: chloroform:hexane=1/6 (v/v), triethylamine 0.1% added} was performed to obtain 13.4 g of a white cake.

¹H-NMR (270 MHz/CDCl3):

δ(ppm)=0.95 [t, 6H], 1.40 [m, 4H], 1.69 [m, 4H], 2.77 [t, 4H], 7.25 [m, 4H], 7.85 [m, 3H]

### SYNTHESIS EXAMPLE 22

### Synthesis of N-biphenylcarbazole Compound

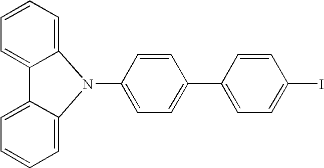

Into a 2 L three-necked flask was added 97.4 g of 4,4′-diiodebiphenyl, 66.3 g of potassium carbonate, 13.4 g of carbazole, 0.36 g of palladium acetate and 780 g of dehydrated toluene. The pressure in the flask was reduced down to 40 mmHg and the pressure was recovered with argon, and this operation was repeated three times, to effect purging with argon. The temperature was raised to 70 to 75° C. and 11.6 ml of a 10% toluene solution of tri(t-butyl)phosphine was weighed by a syringe and added to the flask. The temperature was raised to 105 to 107° C. and reacted under ref lux for 16 hours. The reaction mass was cooled down to room temperature, then, filtrated and the cake was washed with 100 ml of toluene. The filtration bottle was changed, then, the cake was washed three times with 150 ml of ion exchanged water. The cake was transferred to a 500 ml eggplant-shaped flask and dried at 80° C. and 3 mmHg to obtain 52.9 g of solid. Separately, the filtrate was concentrated in a 500 ml eggplant-shaped flask and dried to solid finally at 80° C. and 3 mmHg to obtain 41.4 g of solid. To this solid was added 200 g of toluene and dissolved under reflux, then, cooled down to room temperature to cause deposition of a crystal. The crystal was filtrated and washed with 100 ml of toluene, then, dried at 80° C. and 3 mmHg to obtain 24.9 g of a crystal. 52.9 g of the solid and 24.9 g of the crystal were combined and purified twice by silica gel chromatography {developing solvent: chloroform:hexane=1/5 (v/v), triethylamine 0.1% added} to obtain 22.4 g of white solid. To this solid was added 72 g of chloroform and 72 g of hexane and the mixture was stirred under reflux for 1 hour and cooled down to room temperature, then, filtrated and the cake was washed with 50 ml of hexane. The cake was dried at 80° C. and 3 mmHg to obtain 21.3 g of a crystal. This cake was transferred to a 500 ml eggplant-shaped flask and 60 g of toluene was added to this and the mixture was stirred at 80 to 85° C. for 1 hour. The solution was cooled down to room temperature, then, filtrated and the cake was washed with 50 ml of toluene. This cake was transferred to a 200 ml eggplant-shaped flask and dried at 80 to 85° C. and 3 mmHg to obtain 16.76 g of a white crystal.

¹H-NMR (270 MHz/THF d8):

δ(ppm)=7.22 [m, 2H], 7.37 [m, 4H], 7.53 [d, 2H], 7.68 [d, 2H], 7.86 [m, 4H], 8.12 [d, 2H]

### SYNTHESIS EXAMPLE 23

### Synthesis of Compound L

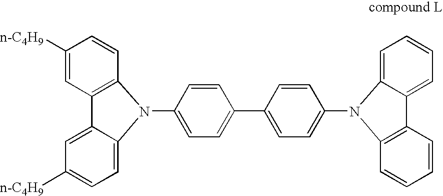

Into a 200 mL three-necked flask was added 8.02 g of the iodobiphenyl compound synthesized in Synthesis Example 22, 7.46 g of potassium carbonate, 6.54 g of 3,6-di-n-butylcarbazole synthesized in Synthesis Example 21, 0.081 g of palladium acetate and 120 g of dehydrated toluene. The pressure in the flask was reduced down to 40 mmHg and the pressure was recovered with argon, and this operation was repeated three times, to effect purging with argon. The temperature was raised to 70 to 75° C. and 2 ml of a 10% toluene solution of tri(t-butyl)phosphine was weighed by a syringe and added to the flask. The temperature was raised to 105 to 107° C. and reacted under ref lux for 16 hours. The reaction mass was cooled down to room temperature, then, filtrated and the cake was washed with 100 ml of toluene. The filtrate was concentrated by a 500 ml eggplant-shaped flask and dried to solid finally at 80° C. and 3 mmHg to obtain 12.54 g of solid. Purification by silica gel chromatography {developing solvent: chloroform:hexane=1/5 (v/v), triethylamine 0.1% added} was performed to obtain 9.51 g of white solid. This is called compound L.

¹H-NMR (270 MHz/CDCl₃):

δ(ppm)=0.97 [t, 6H], 1.44 [m, 4H], 1.73 [m, 4H], 2.82 [t, 4H], 7.29 [m, 4H], 7.47 [m, 6H], 7.87 [d, 4H], 7.92 [m, 6H], 8.17 [d, 2H]

### EXAMPLE 1

### Synthesis of Compound M

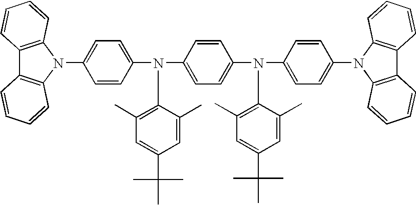

Into a 300 mL three-necked flask was added 18.47 g of N,N′-bis(4-bromophenyl)-N,N′-bis(4-t-butyl-2,6-dimethylphenyl)-1,4-phenylenediamine, 20.73 g of potassium carbonate, 12.54 g of carbazole, 0.11 g of palladium acetate and 185 g of dehydrated toluene. The pressure in the flask was reduced down to 40 mmHg and the pressure was recovered with argon, and this operation was repeated three times, to effect purging with argon. The temperature was raised to 70 to 75° C. and 2.8 ml of a 10% toluene solution of tri(t-butyl)phosphine was weighed by a syringe and added to the flask. The temperature was raised to 105 to 107° C. and reacted under reflux for 12 hours. The reaction mass was cooled down to room temperature, then, filtrated and the cake was washed with 250 ml of toluene. The filtrate was concentrated by a 500 ml eggplant-shaped flask and dried to solid finally at 80° C. and 3 mmHg to obtain 28.32 g of solid. This solid was transferred to a 1 L eggplant-shaped flask and 100 g of toluene and 200 g of ethanol were added and the mixture was stirred under reflux for 1 hour. Next, the solution was cooled down to room temperature and filtrated. The cake was washed with 50 ml of ethanol, then, transferred to a 200 ml eggplant-shaped flask and dried by an evaporator. 21.06 of a dry cake was obtained. Purification by silica gel chromatography {developing solvent: chloroform:hexane=1/3 (v/v), triethylamine 0.1% added} was performed to obtain 15.75 g of while solid.

¹H-NMR (300 MHz/CDCl₃):

δ(ppm)=1.35 [s, 18H], 2.16 [brs, 12H], 7.36 [brs, 28H], 8.39 [d, 4H]

### SYNTHESIS EXAMPLE 24

### Synthesis of Compound N

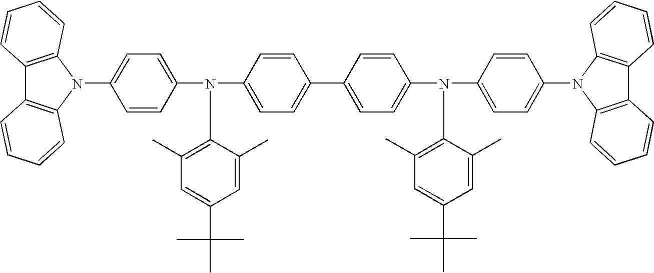

A 100 mL three-necked flask was purged with argon and, Pd(OAc)₂ (22 mg, 0.1 mmol), carbazole (2.51 g, 15 mmol), N,N′-bis(4-bromophenyl)-N,N′-bis(4-t-butyl-2,6-dimethylphenyl)-benzidine (4.07 g, 5 mmol) and K₂CO₃ (4.15 g, 30 mmol) were weighed. Dehydrated toluene (50 mL) was introduced by a syringe, and the mixture was heated up to 70° C. A 10% hexane solution of P(t-Bu)₃ (0.7 ml, 0.25 mmol) was introduced by a syringe, then, the temperature was raised and the mixture was heated under reflux for 17 hours.

After completion of the reaction, the reaction liquid was cooled down to 60° C., and solid was filtrated off. The residue on filter paper was washed with 50 ml of chloroform, then, the filtrate and washing liquid were concentrated and dried to solid. To the concentrated solid was added 50 ml of chloroform and the solid was dissolved under reflux. Next, 50 ml of ethanol was added to cause deposition of a crystal. After cooling down to room temperature, the filtrated cake was washed with 30 ml of ethanol. Drying under reduced pressure at 80° C. was performed to obtain 5.05 g of ash gray solid.

Purification by silica gel chromatography {developing solvent: chloroform:n-hexane (1:2, v/v), 0.1% triethylamine added} was performed to obtain 5.50 g of white solid. Judging from the resulted amount, remaining of chloroform was suspected, thus, the white solid was dissolved at 60° C. in 30 g of toluene, and the solution was concentrated under reduced pressure at 70° C. to obtain 5.30 g of white solid. Further, this solid was dissolved at 80° C. in 100 g of toluene, and 73 g of toluene was distilled off under reduced pressure at 80° C. During distillation, a crystal deposited. 90 g of n-hexane was added to the distillation residue at 80° C. and the mixture was cooled down to room temperature, then, filtrated and the cake was washed with 30 ml of n-hexane. The resultant cake was dried under reduced pressure at 80° C. to obtain white solid (4.48 g, yield: 90.6%).

¹H-NMR (270 MHz/CDCl₃):

δ 1.363 (s, 18H), 2.153 (s, 12H), 7.297 (brs, 32H), 8.132 (d, 4H)

### EXAMPLE 2

### Synthesis of Compound O

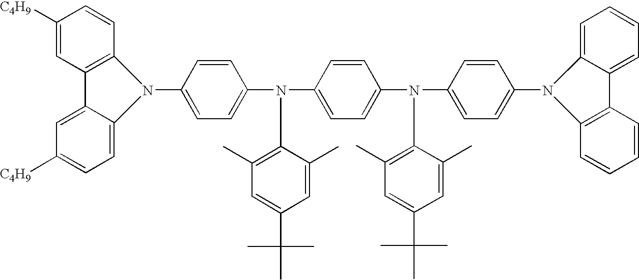

### (1) Synthesis of Dibutylcarbazole Compound

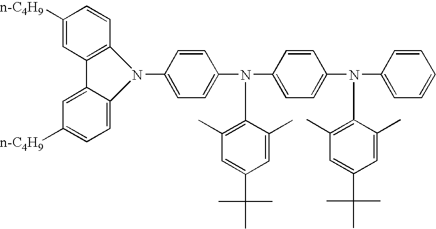

Under an argon atmosphere, into a 200 ml three-necked flask was added 4.36 g of 3,6-di-n-butylcarbazole, 4.98 g of potassium carbonate, 7.92 g of N-phenyl,N′-4-bromophenyl-N,N′-bis(4-t-butyl-2,6-dimethylphenyl)-1,4-phenylenediamine, 0.054 g of palladium acetate and 79.2 g of dehydrated toluene. The pressure in the flask was reduced down to 40 mmHg and the pressure was recovered with argon, and this operation was repeated three times, to effect purging with argon. The temperature was raised to 70 to 75° C. and 1.5 ml of a 10% toluene solution of tri(t-butyl)phosphine was weighed by a syringe and added to the flask, then, reacted for 13 hours at 105 to 107° C. The reaction mass was cooled down to room temperature, then, filtrated and the cake was washed with 30 ml of toluene. The filtrate was concentrated by an evaporator at 80° C. under reduced pressure to obtain 12.2 g of resinous solid. Purification by silica gel chromatography {developing solvent: chloroform:hexane=1/5 (v/v), triethylamine 0.1% added} was performed to obtain 9.60 g of slightly yellowish solid.

### (2) Synthesis of Br Compound

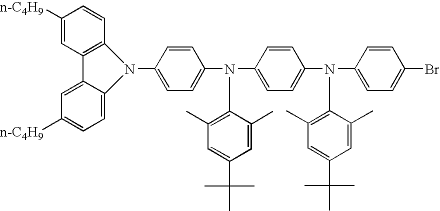

Under an argon atmosphere, 9.60 g of the dibutylcarbazole compound synthesized in (1) and 96.0 g of dehydrated chloroform were added into a 200 ml three-necked flask, and dissolved at room temperature, then, cooled down to −5° C. N-bromosuccinimide was divided into 6 portions and added at −5 to −6° C.: 0.34 g for 5 times every 5 minutes and 0.39 g for sixth time. The mixture was stirred at −5 to 0° C. for 30 minutes, then, the reaction mass was filtrated and the cake was washed with 30 ml of chloroform. The filtrate was transferred to a 300 ml dropping funnel and washed with 50 ml of 2% sodium thiosulfate water. An oil layer was washed twice with 50 ml of ion exchanged water, then, transferred to a 300 ml eggplant-shaped flask and concentrated by an evaporator under reduced pressure. The product was dried to solid finally at 80° C. and 3 mm Hg to obtain 13.69 g of resinous solid. Purification by silica gel chromatography {developing solvent: chloroform:hexane=1/5 (v/v), triethylamine 0.1% added} was performed to obtain 7.78 g of slightly yellowish solid. To the solid was added 30 ml of hexane and 20 ml of ethanol and the mixture was stirred under reflux for 1 hour and cooled down to room temperature, then, filtrated. The wet cake was washed with 30 ml of hexane/ethanol, then, dried at 80° C. and 3 mmHg to obtain 7.77 g of a dry cake.

1H-NMR (270 MHz/THF-d8):

δ(ppm)=0.91 [t, 6H], 1.30 [m, 26H], 1.57 [m, 12H], 2.67 [t, 4H], 6.67 [d, 2H], 7.00 [m, 16H], 7.50 [d, 2H], 7.79 [s, 2H]

### (3) Synthesis of Compound O

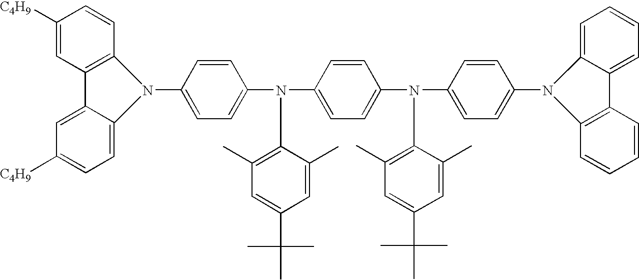

Under an argon atmosphere, into a 200 ml three-necked flask was added 7.50 g of the Br compound synthesized in (2), 4.42 g of potassium carbonate, 2.68 g of carbazole, 0.036 g of palladium acetate and 75.0 g of dehydrated chloroform. The pressure in the flask was reduced down to 40 mmHg and the pressure was recovered with argon, and this operation was repeated three times, to effect purging with argon. The temperature was raised to 70 to 75° C. and 1.0 ml of a 10% toluene solution of tri(t-butyl)phosphine was weighed by a syringe and added to the flask, then, reacted for 8 hours at 105 to 107° C. The reaction mass was cooled down to room temperature, then, filtrated and the cake was washed with 30 ml of toluene. The filtrate was concentrated by an evaporator at 80° C. under reduced pressure to obtain 9.32 g of resinous solid. Purification by silica gel chromatography {developing solvent: chloroform:hexane=1/5 (v/v), triethylamine 0.1% added} was performed to obtain 7.19 g of slightly yellowish solid.

¹H-NMR (270 MHz/THF-d8):

δ(ppm)=0.82 [t, 6H], 1.23 [m, 26H], 1.57 [m, 12H], 2.67 [t, 4H], 7.05 [d, 18H], 7.50 [m, 8H], 7.79 [s, 2H], 7.99 [d, 2H]

### EXAMPLE 3

### Synthesis of Compound P

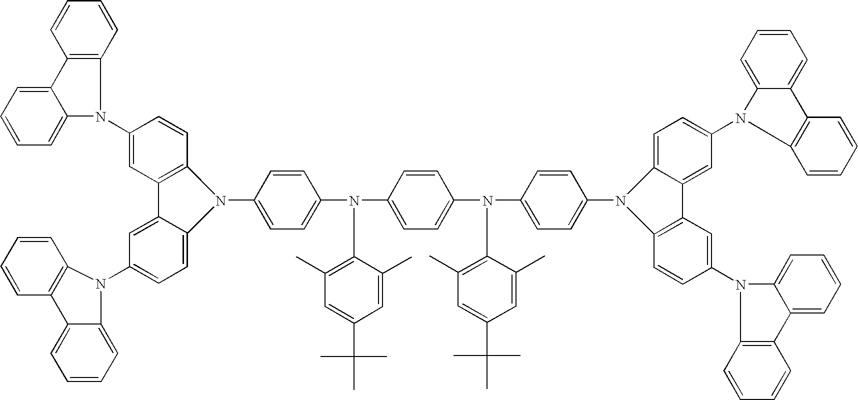

(1) Protection of Amino Group of 3,6-dibromocarbazole

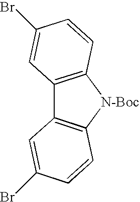

Under a nitrogen atmosphere, into a 1 L three-necked flask was added 93.4 g of 3,6-dibromocarbazole and 1.76 g of 4-dimethylaminopyridine, then, 467 ml of dehydrated tetrahydrofuran was added to dissolve them. 69.0 g of di-t-butyl dicarbonate was weighed into a dropping funnel and dropped over 1.5 hours at 20 to 23° C. under water cool. At the same temperature, the mixture was stirred for 1 hour. The reaction liquid was transferred to a 1 L eggplant-shaped flask and tetrahydrofuran was distilled off by an evaporator at 60° C. under reduced pressure. A crystal deposited during concentration. Next, the crystal was dried at 70° C. and 3 mmHg to obtain 122.4 g of a coarse cake. To the coarse cake was added 250 g of ethanol and the mixture was stirred for 1 hour under reflux and cooled down to room temperature, then, filtrated. The wet cake was washed twice with 150 g of ethanol. To this wet cake was added 250 g of ethanol, and the same purification operation as for the coarse cake was conducted again, then, dried at 80° C. and 3 mmHg. 117.9 g of a slightly yellowish dry cake was obtained.

1H-NMR (270 MHz/CDCl₃):

δ(ppm)=1.75 [s, 9H], 7.57 [d, 2H], 8.03 [s, 2H], 8.16 [d, 2H]

### (2) Synthesis of N-Boc Protected Carbazole Trinuclear Compound

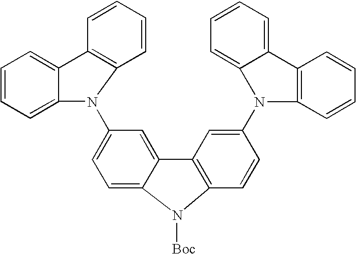

Under an argon atmosphere, into a 200 ml three-necked flask was added 8.50 g of the Boc compound synthesized in (1), 16.58 g of potassium carbonate, 10.03 g of carbazole, 0.09 g of palladium acetate and 85.0 g of dehydrated toluene. The pressure in the flask was reduced down to 40 mmHg at room temperature and the pressure was recovered with argon, and this operation was repeated three times, to purge an atmosphere in the flask with argon. The temperature was raised to 70 to 75° C. and 2.3 ml of a 10% toluene solution of tri(t-butyl)phosphine was weighed by a syringe and added to the flask, then, reacted for 36 hours at 105 to 107° C. The reaction liquid was filtrated, then, the cake was washed with 100 ml of toluene and the filtrate was concentrated by an evaporator at 70° C. under reduced pressure. 15.6 g of solid solidified in the form of resin was obtained. Purification by silica gel chromatography {developing solvent: chloroform:hexane 1/5 (v/v), triethylamine 0.1% added} was performed twice to obtain 4.94 g of a white cake.

¹H-NMR (270 MHz/CDCl₃):

δ(ppm)=1.86 [s, 9H], 7.29 [m, 4H], 7.39 [m, 8H], 7.71 [d, 2H], 8.15 [m, 6H], 8.60 [d, 2H]

### (3) Releasing of Protective Group of Carbazole Trinuclear Compound

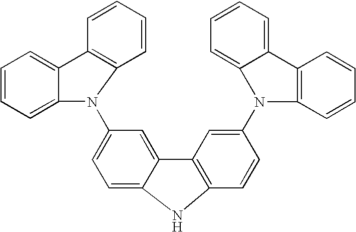

Under an argon atmosphere, into a 100 ml three-necked flask was added 4.90 g of the Boc compound synthesized in (2) and 66 ml of a 1 mol/L tetrahydrofuran solution of tetrabutylammonium fluoride. The mixture was heated and reacted for 36 hours under ref lux. The solution was cooled down to room temperature, then, the reaction mass was transferred to a 200 ml eggplant-shaped flask and concentrated by an evaporator at 60° C. under reduced pressure to obtain 37.5 g of a concentrate. Purification by silica gel chromatography {developing solvent: chloroform:hexane=1/2 (v/v), triethylamine 0.1% added} was performed to obtain 4.37 g of a white cake. To the cake was added 20 g of methanol and the mixture was stirred for 1 hour under reflux and cooled down to room temperature, then, filtrated. The wet cake was washed with 10 ml of methanol, then, dried at 80° C. and 3 mmHg to obtain 3.86 g of a dry cake.

¹H-NMR (270 MHz/CDCl₃):

δ(ppm)=7.28 [m, 4H], 7.39 [m, 8H], 7.84 [m, 4H], 8.16 [m, 6H], 8.38 [brs, 1H]

### (4) Synthesis of Compound P

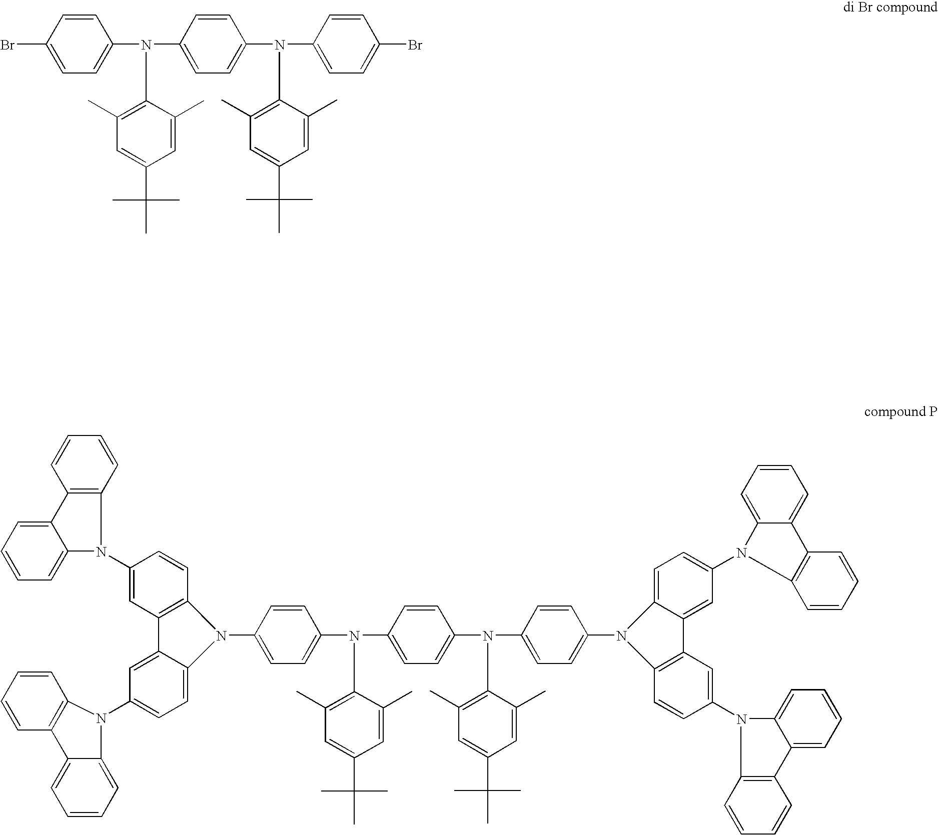

Under an argon atmosphere, into a 100 ml three-necked flask was added 2.49 g of the carbazole trinuclear compound synthesized in (3), 1.66 g of potassium carbonate, 1.48 g of di Br compound (N-phenyl,N′-4-bromophenyl)-N,N′-bis(4-t-butyl-2,6-dimethyl phenyl)-1,4-phenylenediamine), 0.009 g of palladium acetate and 29.6 g of dehydrated toluene. The pressure in the flask was reduced down to 40 mmHg at room temperature and the pressure was recovered with argon, and this operation was repeated three times, to purge an atmosphere in the flask with argon. The temperature was raised to 70 to 75° C. and 0.5 ml of a 5% toluene solution of tri(t-butyl)phosphine was weighed by a syringe and added to the flask, then, reacted for 35 hours at 105 to 107° C. The reaction liquid was filtrated, then, the cake was washed with 50 ml of chloroform and the filtrate was concentrated by an evaporator at 80° C. under reduced pressure. 4.03 g of solid in the form of resin was obtained. Purification by silica gel chromatography {developing solvent: chloroform:hexane=1/2 (v/v), triethylamine 0.1% added} was performed to obtain 2.15 g of a slightly yellowish cake. To the cake was added 30 g of hexane and the mixture was stirred for 1 hour under reflux and cooled down to room temperature, then, filtrated. The wet cake was washed with 10 ml of hexane, then, dried at 80° C. and 3 mmHg to obtain 2.10 g of a dry cake of compound P

¹H-NMR (270 MHz/THF-d8):

δ(ppm) 1.40 [s, 18H], 2.23 [s, 12H], 7.24 [m, 12H], 7.37 [m, 24H], 7.62 [m, 12H], 8.16 [d, 8H], 8.47 [s, 4H]

### SYNTHESIS EXAMPLE 25

### Synthesis of Polymer Compound 3

Compound E (5.25 g), N,N′-bis(4-bromophenyl)-N,N′-bis(4-t-butyl-2,6-dimethylphenyl)-benzidine (3.06 g) and 2,2′-bipyridyl (5.3 g) were dissolved in 226 mL of dehydrated tetrahydrofuran, then, nitrogen was bubbled through the solution to purge an atmosphere in the system with nitrogen. Under a nitrogen atmosphere, to this solution was added bis(1,5-cyclooctadiene)nickel(0){Ni(COD)₂} (9.30 g) and the mixture was heated up to 60° C. and reacted for 3 hours while stirring. After the reaction, this solution was cooled to room temperature (about 25° C.), and dropped into a mixed solution of 25% ammonia water 45 mL/methanol about 230 mL/ion exchanged water about 230 mL and stirred for 1 hour, then, the deposited precipitate was filtrated and dried under reduced pressure for 2 hours, then, dissolved in 400 mL of toluene before conducting filtration, and the filtrate was purified by passing through an alumina column, and about 400 mL of 5.2% hydrochloric acid water was added and the mixture was stirred for 3 hours, then, an aqueous layer was removed. Then, about 400 mL of 4% ammonia water was added and the mixture was stirred form 2 hours, then, an aqueous layer was removed. Further, to an organic layer was added about 400 mL of ion exchanged water and the mixture was stirred for 1 hour, then, an aqueous layer was removed. To an organic layer was added 80 mL of toluene, and the deposited precipitate was collected by decantation and dissolved in 200 ml of toluene, then, this was dropped into about 600 mL of methanol and the mixture was stirred for 1 hour, and the deposited precipitate was filtrated and dried under reduced pressure for 2 hours. The yield of the resultant copolymer (hereinafter, referred to as polymer compound 3) was 4.25 g. The polystyrene-reduced number-average molecular weight and the polystyrene-reduced weight-average molecular weight were Mn=2.5×10⁴ and Mw=8.0×10⁵, respectively (mobile phase: tetrahydrofuran).

### SYNTHESIS EXAMPLE 26

### Synthesis of Polyamine Polymer Compound 4

Under an inert gramosphere, N,N′-bis(4-bromophenyl)-N,N′-bis(4-t-butylphenyl)-1,4-phenylenediamine (1.911 g), N,N′-bis(4-bromophenyl)phenylamine (0.484 g) and 2,2′-bipyridyl (1.687 g) were dissolved in 109 mL of dehydrated tetrahydrofuran previously bubbled with argon. This solution was heated up to 60° C., then, bis(1,5-cyclooctadiene)nickel(0){Ni(COD)₂} (2.971 g) was added and the mixture was stirred and reacted for 5 hours. This solution was cooled to room temperature, and dropped into a mixed solution of 25% ammonia water 14 mL/methanol 109 mL/ion exchanged water 109 mL and stirred for 1 hour, then, the deposited precipitate was filtrated and dried under reduced pressure, and dissolved in 120 mL of toluene. After dissolution, 0.48 g of radiorite was added and the mixture was stirred for 30 minutes, and un-dissolved substances were filtrated. The resultant filtrate was purified through an aluminum column. Next, 236 mL of 4% ammonia water was added and the mixture was stirred for 2 hours, then, an aqueous layer was removed. Further, to an organic layer was added about 236 mL of ion exchanged water, and the mixture was stirred for 1 hour, then, an aqueous layer was removed. Thereafter, an organic layer was poured into 376 mL of methanol and the mixture was stirred for 0.5 hours, and the deposited precipitate was filtrated and dried under reduced pressure. The yield of the resultant polymer (hereinafter, referred to as polymer compound 4) was 1.54 g. The polystyrene-reduced number-average molecular weight and the polystyrene-reduced weight-average molecular weight were Mn=7.4×10³ and Mw=7.6×10⁴, respectively.

### SYNTHESIS EXAMPLE 27

### Synthesis of Polymer Compound 5

22.5 g of compound E and 17.6 g of 2,2′-bipyridyl were charged into a reaction vessel, then, an atmosphere in the reaction system was purged with a nitrogen gas. To this was added 1500 g of tetrahydrofuran (dehydrated solvent) deaerated by previous bubbling with an argon gas. Next, to this mixed solution was added 31 g of bis(1,5-cyclooctadiene)nickel(0) and the mixture was stirred at room temperature for 10 minutes, then, reacted at 60° C. for 3 hours. The reaction was conducted in a nitrogen gas atmosphere.

After the reaction, this reaction solution was cooled, then, into this solution was poured a mixed solution of 25% ammonia water 200 ml/methanol 900 ml/ion exchanged water 900 ml, and the mixture was stirred for about 1 hour. Next, the produced precipitate was filtrated and recovered. This precipitate was dried under reduced pressure, then, dissolved in toluene. This toluene solution was filtrated to remove unnecessary substances, then, this toluene solution was purified by passing through a column filled with alumina. Next, this toluene solution was washed with a 1 N hydrochloric acid aqueous solution, and allowed to stand still to case liquid separation, then, the toluene solution was recovered. Next, this toluene solution was washed with about 3% ammonia water, and allowed to stand still to case liquid separation, then, the toluene solution was recovered. Next, this toluene solution was washed with ion exchanged water, and allowed to stand still to case liquid separation, then, the toluene solution was recovered. Next, this toluene solution was poured into methanol to produce a precipitate again.

Then, the produced precipitate was recovered, and washed with methanol, then, this precipitate was dried under reduced pressure to obtain 6.0 g of a polymer. This polymer is called polymer compound 5. The resultant polymer compound 5 had a polystyrene-reduced weigh-average molecular weight of 8.2×10⁵ and a polystyrene-reduced number-average molecular weight of 1.0×10⁵.

### SYNTHESIS EXAMPLE 28

### Synthesis of Polymer Compound 6

2,7-dibromo-9,9-dioctylfluorene (26 g, 0.047 mol), 2,7-dibromo-9,9-diisopentylfluorene (5.6 g, 0.012 mol) and 2,2′-bipyridyl (22 g, 0.141 mol) were dissolved in 1600 mL of dehydrated tetrahydrofuran, then, nitrogen was bubbled through the solution to purge an atmosphere in the reaction system with nitrogen. Under a nitrogen atmosphere, to this solution was added bis(1,5-cyclooctadiene)nickel(0){Ni(COD)₂} (40 g, 0.15 mol) and the mixture was heated up to 60° C. and reacted for 8 hours. After the reaction, this solution was cooled to room temperature (about 25° C.), and dropped into a mixed solution of 25% ammonia water 200 mL/methanol 1200 mL/ion exchanged water 1200 mL and stirred for 3 hours, then, the deposited precipitate was filtrated and air-dried. Thereafter, the dried precipitate was dissolved in 1100 mL of toluene before conducting filtration, and the filtrate was dropped into 3300 mL of methanol and the mixture was stirred for 30 minutes. The deposited precipitate was filtrated and washed with 1000 mL of methanol, then, dried under reduced pressure for 5 hours. The yield of the resultant polymer was 20 g. This polymer is called polymer compound 6. The polystyrene-reduced average molecular weights of polymer compound 6 were Mn=4.6×10⁴ and Mw=1.1×10⁵.

### <Preparation of Light Emitting Polymer Solution Composition>

Polymer compounds as light emitting polymers as shown in Table 1 were dissolved in a proportion of 1 wt % in toluene, further, additives of kinds and addition amounts shown in Table 1 were mixed and dissolved. In Example 6 showing incomplete dissolution, chloroform was additionally added as a solvent. Then, the mixture was filtrated through a teflon (registered trademark) filter of 0.2 micron size to prepared an application solution.

TABLE 1

| | | | Addition | |
|---|---|---|---|---|
| | Composition of | | amount | Maximum |
| | light emitting | Kind of | (parts by | efficiency |
| | polymer | additive | weight *1) | (Cd/A) |
| Example 4 | Polymer | Compound F | 40 | 4 |
| | compound | | | |
| | 1/2 = 50/50 | | | |
| Example 5 | Polymer | Compound F | 100 | 3 |
| | compound | | | |
| | 1/2 = 50/50 | | | |
| Example 6 | Polymer | DCBP | 10 | 2.5 |
| | compound | (*2) | | |
| | 1/2 = 50/50 | | | |
| Example 7 | Polymer | DCBP | 20 | 3.5 |
| | compound | | | |
| | 1/2 = 50/50 | | | |
| Example 8 | Polymer | DCBP | 40 | 4.2 |
| | compound | | | |
| | 1/2 = 50/50 | | | |
| Example 9 | Polymer | DCBP | 100 | 3.2 |
| | compound | | | |
| | 1/2 = 50/50 | | | |
| Example 10 | Polymer | DCBP | 40 | 2.3 |
| | compound | | | |
| | 1/3 = 50/50 | | | |
| Example 11 | Polymer | Compound G | 80 | 2 |
| | compound | | | |
| | 1/3 = 50/50 | | | |
| Example 12 | Polymer | Compound H | 40 | 1.7 |
| | compound | | | |
| | 1/3 = 50/50 | | | |
| Example 13 | Polymer | Compound I | 40 | 2.1 |
| | compound | | | |
| | 1/3 = 50/50 | | | |
| Example 14 | Polymer | Compound J | 40 | 2.4 |
| | compound | | | |
| | 1/3 = 50/50 | | | |
| Example 15 | Polymer | Compound K | 40 | 2.4 |
| | compound | | | |
| | 1/3 = 50/50 | | | |
| Example 16 | Polymer | Compound L | 40 | 2.4 |
| | compound | | | |
| | 1/3 = 50/50 | | | |
| Example 17 | Polymer | Compound M | 40 | 1.5 |
| | compound | | | |
| | 1/3 = 50/50 | | | |
| Example 18 | Polymer | Compound N | 40 | 2.0 |
| | compound | | | |
| | 1/3 = 50/50 | | | |
| Example 19 | Polymer | Compound O | 40 | 2.2 |
| | compound | | | |
| | 1/3 = 50/50 | | | |
| Example 20 | Polymer | Compound P | 40 | 2.8 |
| | compound | | | |
| | 1/3 = 50/50 | | | |
| Example 21 | Polymer | Compound N | 40 | 1.6 |
| | compound1 = 100 | | | |
| Example 22 | Polymer | Compound N | 80 | 1.6 |
| | compound1 = 100 | | | |
| Comparative | Polymer | — | 0 | 2 |
| Example 1 | compound | | | |
| | 1/2 = 50/50 | | | |
| Comparative | Polymer | — | 0 | 1.1 |
| Example 2 | compound | | | |
| | 1/3 = 50/50 | | | |
| Comparative | Polymer | — | 0 | 0.25 |
| Example 3 | compoundl = 100 | | | |

*1 addition amount of additive based on 100 parts by weight of the total
weight of light emitting polymer
*2 DCBP 4,4′-bis(9-carbazoyl)-biphenyl of the following formula
(manufactured by Dojin Kagaku Kenkyusho K.K.)

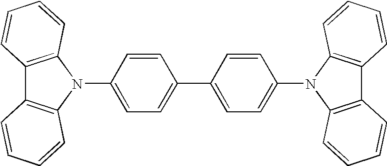

### <Manufacturing and Evaluation of Device>

On a glass base plate carrying an IO membrane having a thickness of 150 nm provided by a sputtering method, a film with a thickness of 70 nm was formed using a solution of poly(ethylenedioxythiophene)/polystyrenesulfonic acid (Baytron, manufactured by Bayer) by spin coat, and dried on a hot plate at 200° C. for 10 minutes. Next, using the prepared light emitting polymer application solution, a film with a thickness of about 70 nm was formed by spin coat at a revolution of 1400 rpm. Further, this was dried at 90° C. for 1 hour under reduced pressure, then, lithium fluoride was vapor-deposited with a thickness of 4 nm as a cathode buffer layer and calcium was vapor-deposited with a thickness of 5 nm and aluminum was vapor-deposited with a thickness of 100 nm as a cathode, manufacturing a polymer LED. The degree of vacuum in vapor deposition was always from 1 to 9×10⁻⁵ Torr. By applying voltage step-by-step on the resultant device having an emitting par of 2 mm×2 mm (area: 4 mm²), the brilliance of EL light emission from the light emitting polymer was measured, thereby obtaining current efficiency value. The maximum values of the resultant device current efficiency are shown in Table 1. Devices using a 50/50 mixture of polymer compound 1 and polymer compound 2 as a light emitting polymer showed EL emission of λmax=479 nm and devices using a 50/50 mixture of polymer compound 1 and polymer compound 3 showed EL emission of λmax=460 nm. In comparison with polymer light emitting devices in comparative examples not containing compounds F to N, DCBP, polymer light emitting devices manufactured using application solutions in Examples 4 to 22 containing compounds F to N, DCBP showed remarkable improvement in efficiency.

### Manufacturing of Polyamine Hole Transport Layer 1

On a glass base plate carrying an IO membrane having a thickness of 150 nm provided by a sputtering method, a film was formed by spin coat using a solution of PEDOT: poly(ethylenedioxythiophene)/polystyrenesulfonic acid (Baytron, manufactured by Starkvitek), and dried on a hot plate at 200° C. for 10 minutes, to form a PEDOT layer as a hole injection layer with a thickness of 50 nm. Next, a 1 wt % toluene solution of polyamine polymer compound 4 was applied at a revolution of 500 rpm. Thereafter, the base plate was baked at 200° C. for 10 minutes in a nitrogen atmosphere to manufacture polyamine hole transport layer 1.

### Manufacturing of Polyamine Hole Transport Layer 2

In a 1 wt % toluene solution of polyamine polymer compound 4, dipentaerythritol hexaacrylate (KAYARAD DPHA manufactured by Nippon Kayaku Co., LTd.) was mixed and dissolved as a cross-linking agent in a proportion of 25 wt % based on the polymer compound, and the resultant solution was applied at a revolution of 500 rpm on a glass base plate carrying an IO membrane having a thickness of 150 nm provided by a sputtering method. Thereafter, the base plate was baked at 300° C. for 20 minutes in a nitrogen atmosphere to manufacture polyamine hole transport layer 2 having a thickness of 50 nm.

### <Preparation of Light Emitting Polymer Solution Composition>

Polymer compounds as light emitting polymers as shown in Table 2, further, additives and coloring matters of kinds and addition amounts shown in Table 2 were mixed and dissolved in toluene. Then, the mixture was filtrated through a teflon (registered trademark) filter of 0.2 micron size to prepared an application solution.

### <Manufacturing and Evaluation of Device>

Next, a film with a thickness of about 70 nm was formed by spin coat using the prepared light emitting polymer application solution. Further, this was dried at 90° C. under reduced pressure for 1 hour, then, lithium fluoride was vapor-deposited with a thickness of 4 nm as a cathode buffer layer and calcium was vapor-deposited with a thickness of 5 nm and aluminum was vapor-deposited with a thickness of 100 nm as a cathode, manufacturing a polymer LED. The degree of vacuum in vapor deposition was always from 1 to 9×10⁻⁵ Torr. By applying voltage step-by-step on the resultant device having an emitting par of 2 mm×2 mm (area: 4 mm²), the brilliance of EL light emission from the light emitting polymer was measured, thereby obtaining current efficiency value. The maximum values of the resultant device current efficiency are shown in Table 2.

### Manufacturing of Polyamine Hole Transport Layer 1

On a glass base plate carrying an IO membrane having a thickness of 150 nm provided by a sputtering method, a film was formed by spin coat using a solution of PEDOT: poly(ethylenedioxythiophene)/polystyrenesulfonic acid (Baytron, manufactured by Starkvitek), and dried on a hot plate at 200° C. for 10 minutes, to form a PEDOT layer as a hole injection layer with a thickness of 50 nm. Next, a 1 wt % toluene solution of polyamine polymer compound 4 was applied at a revolution of 500 rpm. Thereafter, the base plate was baked at 200° C. for 10 minutes in a nitrogen atmosphere to manufacture polyamine hole transport layer 1.

### Manufacturing of Polyamine Hole Transport Layer 2

In a 1 wt % toluene solution of polyamine polymer compound 4, dipentaerythritol hexaacrylate (KAYARAD DPHA manufactured by Nippon Kayaku Co., LTd.) was mixed and dissolved as a cross-linking agent in a proportion of 25 wt % based on the polymer compound, and the resultant solution was applied at a revolution of 500 rpm on a glass base plate carrying an IO membrane having a thickness of 150 nm provided by a sputtering method. Thereafter, the base plate was baked at 300° C. for 20 minutes in a nitrogen atmosphere to manufacture polyamine hole transport layer 2 having a thickness of 50 nm.

### <Preparation of Light Emitting Polymer Solution Composition>

Polymer compounds as light emitting polymers as shown in Table 2, further, additives and coloring matters of kinds and addition amounts shown in Table 2 were mixed and dissolved in toluene. Then, the mixture was filtrated through a teflon (registered trademark) filter of 0.2 micron size to prepared an application solution.

### <Manufacturing and Evaluation of Device>

Next, a film with a thickness of about 70 nm was formed by spin coat using the prepared light emitting polymer application solution. Further, this was dried at 90° C. under reduced pressure for 1 hour, then, lithium fluoride was vapor-deposited with a thickness of 4 nm as a cathode buffer layer and calcium was vapor-deposited with a thickness of 5 nm and aluminum was vapor-deposited with a thickness of 100 nm as a cathode, manufacturing a polymer LED. The degree of vacuum in vapor deposition was always from 1 to 9×10⁻⁵ Torr. By applying voltage step-by-step on the resultant device having an emitting par of 2 mm×2 mm (area: 4 mm²), the brilliance of EL light emission from the light emitting polymer was measured, thereby obtaining current efficiency value. The maximum values of the resultant device current efficiency are shown in Table 2.

TABLE 2

| | | | | | | Addition | |
|---|---|---|---|---|---|---|---|
| | | | | | | amount | |
| | | | | | | of | |
| | | | | Addi- | | color- | |
| | | | | tion | | ing | |
| | Kind of | | | amount | Kind | matter | |
| | polyamine | | | (parts | of | (parts | |
| | hole | Composition of | | by | color- | by | Maximum |
| | transport | light emitting | Kind of | weight | ing | weight | efficiency |
| | layer | polymer | additive | *3) | matter | *4) | (Cd/A) |
| Example | Polyamine | Polymer | DCBP | 160 | — | — | 3.5 |
| 23 | hole | compound1/3 = | | | | | |
| | transport | 72/25 | | | | | |
| | layer 1 | | | | | | |
| Example | Polyamine | Polymer | DCBP | 160 | — | — | 4.5 |
| 24 | hole | compound5 = | | | | | |
| | transport | 100 | | | | | |
| | layer 1 | | | | | | |
| Example | Polyamine | Polymer | Compound | 160 | — | — | 4.3 |
| 25 | hole | compound5 = | J | | | | |
| | transport | 100 | | | | | |
| | layer 1 | | | | | | |
| Example | Polyamine | Polymer | DCBP | 160 | — | — | 4.9 |
| 26 | hole | compound1/3 = | | | | | |
| | transport | 72/25 | | | | | |
| | layer 2 | | | | | | |
| Example | Polyamine | Polymer | DCBP | 160 | — | — | 5.0 |
| 27 | hole | compound5 = | | | | | |
| | transport | 100 | | | | | |
| | layer 2 | | | | | | |
| Example | Polyamine | Polymer | Compound | 160 | — | — | 4.6 |
| 28 | hole | comnpound5 = | L | | | | |
| | transport | 100 | | | | | |
| | layer 2 | | | | | | |
| Example | Polyamine | Polymer | Compound | 160 | — | — | 4.6 |
| 29 | hole | compound5 = | L | | | | |
| | transport | 100 | | | | | |
| | layer 2 | | | | | | |
| Example | Polyamine | Polymer | DCBP | 160 | ADS07 | 2 | 6.5 |
| 30 | hole | compound6 = | | | 8GE | | |
| | transport | 100 | | | | | |
| | layer 2 | | | | | | |
| Example | Polyamine | Polymer | Compound | 160 | — | — | 4.5 |
| 31 | hole | compound5 = | N | | | | |
| | transport | 100 | | | | | |
| | layer 1 | | | | | | |
| Example | Polyamine | Polymer | Compound | 160 | — | — | 4.6 |
| 32 | hole | compound5 = | N | | | | |
| | transport | 100 | | | | | |
| | layer 2 | | | | | | |
| Example | Polyamine | Polymer | Compound | 160 | ADS07 | 2 | 8.1 |
| 33 | hole | conipound6 = | N | | 8GE | | |
| | transport | 100 | | | | | |
| | layer 2 | | | | | | |
| Compar- | Polyamine | Polymer | — | 0 | — | — | 2.9 |
| ative | hole | compound1/3 = | | | | | |
| Example | transport | 72/25 | | | | | |
| 4 | layer 1 | | | | | | |
| Compar- | Only | Polymer | — | 0 | — | — | 2.6 |
| ative | PEDOT | compound1/3 = | | | | | |
| Example | | 72/25 | | | | | |
| 5 | | | | | | | |
| Compar- | Polyamine | Polymer | — | 0 | — | — | 2.4 |
| ative | hole | compound5 = | | | | | |
| Example | transport | 100 | | | | | |
| 6 | layer 1 | | | | | | |
| Compar- | Only | Polymer | — | 0 | — | — | 0.1 |
| ative | PEDOT | compound5 = | | | | | |
| Example | | 100 | | | | | |
| 7 | | | | | | | |
| Compar- | Polyamine | Polymer | — | 0 | AD507 | 2 | 3 |
| ative | hole | compound6 = | | | 8GE | | |
| Example | transport | 100 | | | | | |
| 8 | layer 2 | | | | | | |

*3 addition amount of additive based on 100 parts by weight of the total weight of light emitting
polymer
*4 addition amount of coloring matter based on 100 parts by weight of the total weight of light
emitting polymer and additive
ADS078GE indium complex coloring matter of the following formula manufactured by American
Diesource

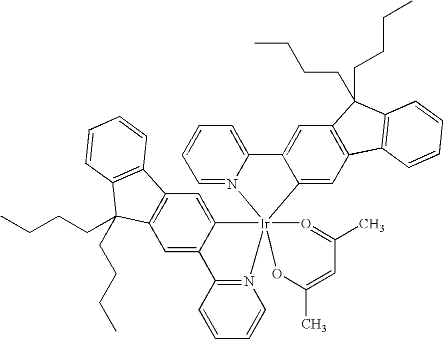

Devices not containing coloring matters in the light emitting polymer application solution showed blue EL light emission of λmax=460 nm, and devices containing coloring matters showed white EL light emission of two peaks of λmax=460 nm and λmax=555 nm. In comparison with polymer light emitting devices in comparative examples not containing compounds J, L, N, DCBP, polymer light emitting devices manufactured using application solutions in Examples 23 to 33 containing compounds J, L, N, DCBP showed remarkable improvement in efficiency.

### INDUSTRIAL APPLICABILITY

By allowing the light emitting polymer composition of the present invention to be contained in a light emitting layer of a light emitting device, the efficiency of the device can be enhanced. Therefore, a polymer LED using the light emitting polymer composition of the present invention can be preferably used in curved or sheet light sources for illumination or backlight of liquid crystal displays, display devices of segment type, flat panel displays of dot matrix, and the like.

1. A light emitting polymer composition comprising a light emitting polymer and a compound selected from the following formulae (1a) to (1d): 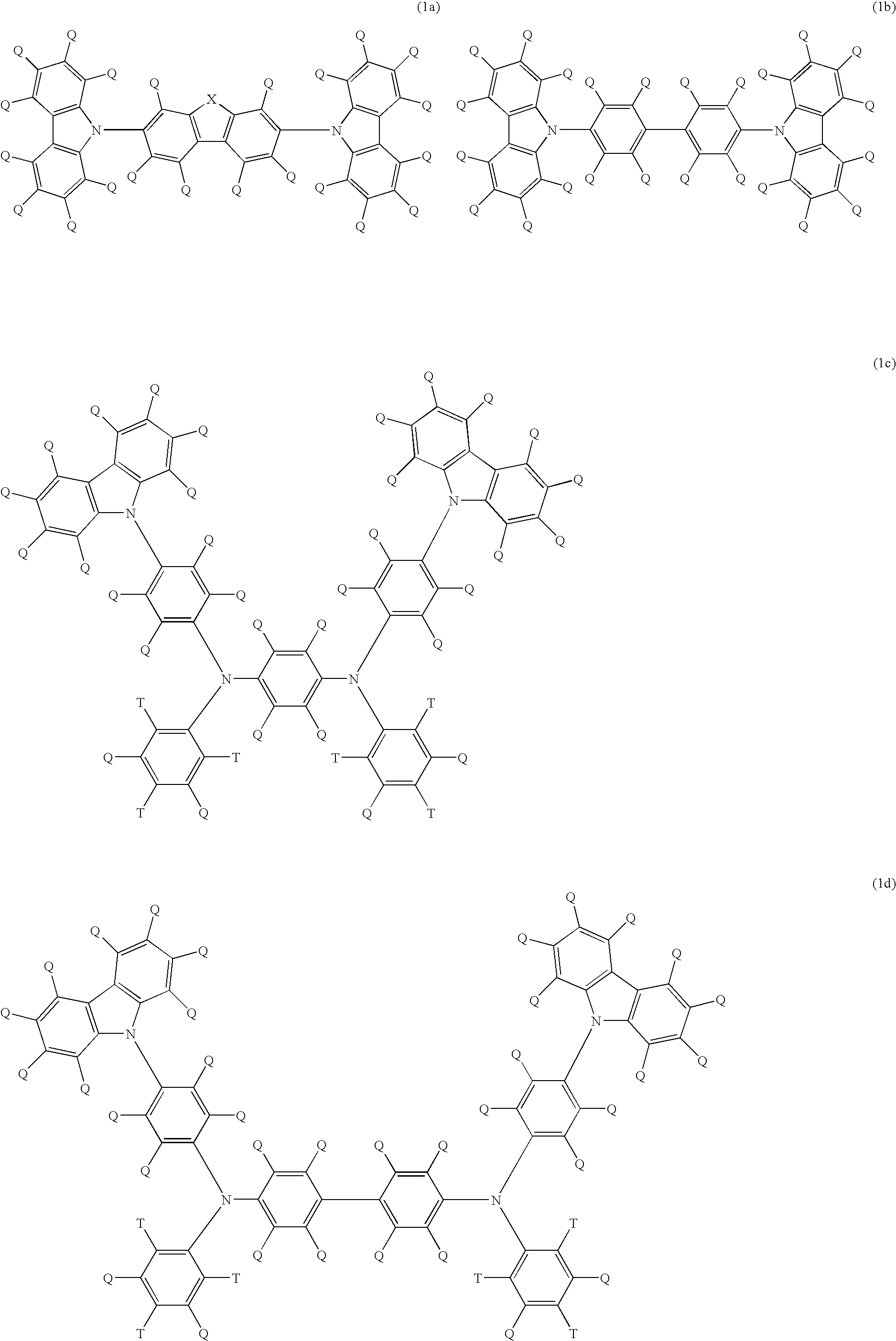
(wherein, X represents an atom or atomic group forming a 5-membered or 6-membered ring together with four carbon atoms on two benzene rings in the formula, Q and T represent each independently a hydrogen atom, halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, substituted silyloxy group, substituted silylthio group, substituted silylamino group, substituted amino group, amide group, acid imide group, acyloxy group, mono-valent heterocyclic group, heteroaryloxy group, heteroarylthio group, cyano group or nitro group, and of them, any two moieties bonding to adjacent carbon atoms may together form a ring. A plurality of Qs and a plurality of Ts may be the same or different, respectively).

2. The light emitting polymer composition according to claim 1, wherein Q and T represent each independently a hydrogen atom or alkyl group.

3. The light emitting polymer composition according to claim 1, wherein T is selected from halogen atoms, alkyl groups, alkyloxy groups, alkylthio groups, aryl groups, aryloxy groups, arylthio groups, arylalkyl groups, arylalkyloxy groups, arylalkylthio groups, alkenyl groups, alkynyl groups, arylalkenyl groups, arylalkynyl groups, substituted silyloxy groups, substituted silylthio groups, substituted silylamino groups, substituted amino groups, amide groups, acid imide groups, acyloxy groups, mono-valent heterocyclic groups, hetero aryloxy groups, hetero arylthio groups, cyano group and nitro group.

4. The light emitting polymer composition according to claim 1, wherein the light emitting polymer comprises a repeating unit of the following formula (2): 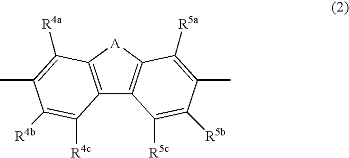
(wherein, A represents an atom or atomic group forming a 5-membered or 6-membered ring together with four carbon atoms on two benzene rings in the formula, R^{4a}, R^{4b}, R^{4c}, R^{5a}, R^{5b} and R^{5c} represent each independently a hydrogen atom, halogen atom, alkyl group, alkyloxy group, alkylthio group, aryl group, aryloxy group, arylthio group, arylalkyl group, arylalkyloxy group, arylalkylthio group, alkenyl group, alkynyl group, arylalkenyl group, arylalkynyl group, acyl group, acyloxy group, amide group, acid imide group, imine residue, substituted amino group, substituted silyl group, substituted silyloxy group, substituted silylthio group, substituted silylamino group, cyano group, nitro group, mono-valent heterocyclic group, heteroaryloxy group, heteroarylthio group, alkyloxycarbonyl group, aryloxycarbonyl group, arylalkyloxycarbonyl group, heteroaryloxycarbonyl group or carboxyl group, and R^{4b} and R^{4c}, and R^{5b} and R^{5c} may together form a ring, respectively).

5. The light emitting polymer composition according to claim 1, wherein the content of a compound selected from the formulae (1a) to (1d) is 0.1 to 10000 parts by weight based on 100 parts by weight of the light emitting polymer.

6. A light emitting polymer solution composition comprising the light emitting polymer composition according to claim 1 and further comprising a solvent.

7. A polymer light emitting device having a light emitting layer between electrodes composed of an anode and a cathode wherein the light emitting layer comprises the light emitting polymer composition according to claim 1.

8. A polymer light emitting device having a light emitting layer between electrodes composed of an anode and a cathode wherein the light emitting layer is formed using the light emitting polymer solution composition according to claim 6.

9. A compound of the above-described formula (1c).

10. The polymer light emitting device according to claim 7, wherein a hole transporting layer made of a polyamine having a repeating unit derived from an aromatic amine is present between electrodes composed of an anode and a cathode.
